(19) United States
(12) Patent Application Publication
(10) Pub. No.: US 2008/0070242 A1
(43) Pub. Date: Mar. 20, 2008

(54) Method for cancer detection and monitoring

(76) Inventors: Linda Pilarski, Spring Lake (CA); Sophia Adamia, Edmonton (CA)

Correspondence Address: Craig K Sherburne, # 35-1011 Canterbury Drive SW, Calgary, AB, T2W 2S8, US

(21) Appl. No.: 11/399,348

(22) Filed: Apr. 7, 2006

Related U.S. Application Data
(60) Provisional application No. 60/669,368, filed on Apr. 8, 2005.

(51) Int. Cl. C12Q 1/68

(52) U.S. Cl. 435/6

(57) ABSTRACT

The present invention provides for methods for detection of cancer or predisposition to cancer comprising detection of specific mutations in genomic DNA encoding the HAS1 protein. The present invention further provides for methods for monitoring of disease progression in a mammalian patient and novel therapeutic methodologies for treatment of disease.

### RELATED APPLICATION

This application claims the benefit of U.S. Provisional Application Ser. No. 60/669,368, filed Apr. 8, 2006, under 35 U.S.C. 119(e). The entire disclosure of the prior application is hereby incorporated by reference.

### FIELD OF THE INVENTION

The present invention pertains to the field of medical diagnostics and in particular to the detection of cancer and predisposition to cancer.

### BACKGROUND OF THE INVENTION

All of the publications, patents and patent applications cited within this application are herein incorporated by reference in their entirety to the same extent as if the disclosure of each individual publication, patent application or patent was specifically and individually indicated to be incorporated by reference in its entirety.

Hyaluronan (HA) and HASs

Current models of carcinogenesis describe cancer as a progression of genetic mutations in a tumour cell mass and these models have contributed to the discoveries of many tumour suppressor genes and potential oncogenes (Hanahan, D. et al. Cell 100:57 (2000)). The progression of genetic mutations can arise from a genetic instability in the cell leading to a loss in replication fidelity, genetic translocations or loss of genetic material. Solid tumours, however, are more than clonal expansions of tumour cells; tumours are heterogeneous and have a complex structure, with Bissell describing a tumour as a unique “organ” formed by “tissues” (Bissell, M. J. et al. Nat Rev Cancer 1:46 (2001)). The cells composing these tissues interact with each other and with other types of cells and exchange information through cell-cell interactions or through interactions with cytokines and the extracellular matrix (ECM) (Bissell, M. J. et al. Nat Rev Cancer 1:46 (2001)). Playing an important role in these interactions, and possibly playing a role in proliferative disease progression as taught in the art and as discovered by the inventors and herein disclosed, is hyaluronic acid.

HA, a non-sulfated negatively charged glycosaminoglycan, is composed of repeating disaccharide units of D-glucorinic acid and N-acetylglucosamine. HA is completely biodegradable by a natural catalytic pathway and is widely distributed in all connective tissue of eukaryotes and in the capsules of group A and C streptococci (Laurent, T. C. et al. FASEBJ6:2397 (1992)). HA is involved in many biological processes such as embryogenesis, cell adhesion and motility, cell growth and differentiation, and angiogenesis (Banerjee, S. D. et al. J Cell Biol 119:643 (1992); Bourguignon, L. Y. et al. J Biol Chem 272:27913 (1997); Lees, V. C. et al. Lab Invest 73:259 (1995); West, D. C. et al. Science 228:1324 (1985)).

HA, which is widely distributed in all connective tissue of eukaryotes, is a water-like molecule; because of this characteristic HA has been regarded as an ideal lubricant of the joints and has been successfully used in the treatment of patients with arthritis (Radin, E. L. et al. Nature 228:377 (1970)) where HA forms a layer between the cartilage surfaces in joints and protects them from frictional damage (Hlavacek, M., J Biomech 26:1151 (1993)). In arthritis, the mechanism forming protective HA layers is disrupted since the concentration of HA itself and molecular weight of the HA molecules are low as compared to normal tissues (Hlavacek, M., J Biomech 26:1151 (1993)). Depletion of HA results in degradation of the ECM and promotes osteoarthritis, a degenerative disease of articular cartilage.

Dramatically increased HA-rich matrix formation has been observed around proliferating and migrating cells during morphogenesis, regeneration and healing. High amounts of HA molecules are synthesized:

1) prior to the mesenchymal cell differentiation and throughout embryonic development, the condensation and differentiation of the mesenchymal cells are accompanied by the spatial distribution of HA in the different regions of the limb bud, (Kosher, R. A. et al. Cell Differ 17:159 (1985); Kosher, R. A. et al. Nature 291:231 (1981); Kosher, R. A. et al. J Embryol Exp Morphol 56:91 (1980)).

2) during brain development around proliferating and migrating neuronal cells, (Verna, J. M. et al. Int J Dev Neurosci 7:389 (1989)), and

3) during formation of heart valves when cushion cells migrate from the endocardium to the myocardium (Camenisch, T. D. et al. J Clin Invest 106:349 (2000)).

HA matrices are removed from the cells after final differentiation at the end of morphogenetic events (Gakunga, P. et al. Development 124:3987 (1997)). Throughout morphogenesis HA creates hydrated pathways, thus facilitating free movement of the cells in this microenvironment. (Gakunga, P. et al. Development 124:3987 (1997)). HA molecules are conducive to cell proliferation and migration, preventing differentiation of cells until sufficient number and appropriate positioning of cells is established, which is essential for the formation of tissues and/or organs (Gakunga, P. et al. Development 124:3987 (1997)). In addition, the formation of hydrated pathways by HA molecules is closely associated with the surface of different types of cells, and these associations promote cell adhesion and aggregation (Sionov, R. V. et al. Adv Cancer Res 71:241 (1997); Lee, V. et al. J Cell Biochem 79:322 (2000)).

The motility of malignant cells is mediated through interactions with HA, which is an important extracellular matrix molecule (Docherty, R. et al. J Cell Sci 92:263 (1989); Ropponen, K. et al. Cancer Res 58:342 (1998); Ruoslahti, E. J Biol Chem 264:13369 (1989); Sherman, L., et al. Curr Opin Cell Biol 6:726 (1994); Zhang, W. et al. Biochem J349:91 (2000)). High or very low levels of HA in the serum of patients with multiple myeloma (MM) correlate with dramatically reduced median survival of these patients (Dahl, I. M. et al. Blood 93:4144 (1999b)). Moreover, HA mediates survival of MM cell lines against dexamethasone-induced apoptosis through IL-6-dependent and -independent autocrine pathways (Vincent, T. et al. Br Haematol 121:259 (2003)). HA also increases intracellular Ca2⁺ levels by binding to CD44, suggesting that HA may activate intracellular signaling through activation of protein kinase C (Fraser, S. P. FEBS Lett. 404:56 (1997); Liu, D. et al. Cell Immunol 174:73 (1996); Milstone, L. M. et al. J Cell Sci 107:3183 (1994)). Also secretion of HA is stimulated by growth factors which activate classical and novel isoform (PKCa) of PKC (Anggiansah, C. L. et al. J Physiol 550:631 (2003)). In addition to its role as an ECM and signaling molecule, HA plays a significant role in the process of mitosis and in the maintenance of cell shape or volume (DeAngelis, P. L., Cell Mol Life Sci 56:670 (1999); Evanko, S. P. et al. Arterioscler Thromb Vasc Biol 19:1004 (1999)).

HA has complex biological effects, especially as related to cancer. Aberrant endogenous production of HA or treatment with exogenous HA in vitro has been shown in multiple model systems to promote cancer cell growth and malignant behavior (Toole, B. P. Glycobiology 12:42R (2003)). HAS1 is a prognostic factor in MM, ovarian and colon cancer (Adamia, S. et al. Blood 102:5211 (2003); Yamada, Y. Clin. Exp. Metastasis 21:57 (2004); Yabushita, H. et al. Oncol. Rep. 12:739 (2004)). Dahl et al. demonstrated that abnormally high or very low levels of HA in the serum of patients with MM correlate with dramatically reduced median survival of these patients (Dahl, I. M. et al. Blood 93:4144 (1999)), confirming the importance of HA synthesis and metabolism in MM. On the other hand, treatment in vivo with exogenous HA can inhibit cancer growth (Herrera-Gayola, A. et al. Exp Mol Pathol 72:179 (2002); Zeng, C. et al. Int J Cancer 77:396)). It is contemplated that multiple mechanisms are involved in either stimulation or inhibition of cancer by HA. To understand the impact of HA in any given model of cancer or in cancer patients themselves, it is necessary to evaluate HA synthesis, HASs and HA receptors.

HA molecules are synthesized by HASs, integral transmembrane proteins with multiple enzymatic activities and a probable pore-like structure (Weigel, P. H. et al. J Biol Chem 272:13997 (1997); Tlapak-Simmons V. L. et al. J Biol Chem. 274:4239 (1999); Heldermon, C. et al. J Biol Chem 276:2037 (2001). Three isoenzymes of HAS, HAS1 (hCh19), HAS2 (hCh8), and HAS3 (hCh16), have been detected in humans thus far. Each isoenzyme of HAS synthesizes different sizes of HA molecules which exhibit different functions (Itano, N. et al. J Biol Chem 274:25085 (1999); Itano, N. et al. J Biol Chem 279:18669-87 (2004)). The role of HAS genes in different types of cancer is well documented (Ichikawa, T. et al. J Invest Dermatol 113:935 (1999); Auvinen, P. K. et al. Int J Cancer 74:477 (1997); Auvinen, P. et al. Am J Pathol 156:529 (2000); Anttila, M. A. et al. Cancer Res 60:150 (2000); Setala, L. P. et al. Br J Cancer 79:1133 (1999); Liu, N. et al. Cancer Res 63:5207 (2001); Simpson, M. A. et al. J Biol Chem 277:10050 (2002); Simpson, M. A. et al. Am J Pathol 161:849 (2002); Kosaki, R. et al. Cancer Res 59:1141 (1999)). Overexpression of HAS proteins promotes growth and/or metastatic development in fibrosarcoma, prostate and mammary carcinoma and the removal of the HA matrix from a migratory cell membrane inhibits cell movement (Simpson, M. A., et al. J Biol Chem 277:10050 (2002); Itano, N. et al. Cancer Res 59:2499 (1999)). Although extensive reports characterize HAS2 and HAS3, little is known about the role of HAS1 in various types of cancers, likely because of the transcripts are of low abundance and/or short lived due to AU-rich elements (ARE) on the 3′ untranslated region of the gene, which are known to control mRNA half life (ARE Dotobase: http://rc.kfshrc,edu.sa/ared/) (Bevilacqua, A. et al. J Cell Physiol 195:356 (2003); Chen, C. Y. et al. Trends Biochem Sci 20:465 (1995)).

Aberrant HAS1 Splice Variant transcripts in MM and WM:

A family of splice variants of HAS1 expressed in MM and Waldenstrom's Macroglobulinemia (WM) has recently been identified (US Patent Application #20050003368). HAS1Va results from complete deletion of exon 4, which leads to a frameshift after the deletion of exon 4 and “insertion” of a premature termination codon (PTC), 56 base pairs (bp) downstream of the deletion (FIG. 1a). HAS1Vb appears to be the result of partial retention of intron 4 (59 bp) at the 5′ end of exon 5 and the deletion of the entire exon 4 (FIG. 1b). These aberrations lead to a frameshift after deletion of exon 4 and harbor a PTC 93 nucleotides downstream of retained intron 4, at the beginning of the exon 5 (FIG. 2). HAS1Vc is similar to HAS1Vb and appears result from retention of 26 bp of intron 4 at the 3′ end of exon 4, causing truncation of the HAS1 transcripts and “insertion” of PTC at the 3′ end of exon 4 (FIG. 1c). For all three variants, the start codon and the entire sequence of the enzymatically active intracellular loop previously described for Xenopus x1HAS1 are present in the aligned cDNA sequences obtained from CD19⁺ B cells, suggesting that they retain the ability to synthesize HA. All three HAS1 splice variants are likely to encode a functional protein, since the enzymatically active central loop of the protein is retained. This was verified by alignment analysis which demonstrated that the conserved amino acids determining the size of HA molecules are retained. The occurrence of a point mutation T/C in HAS1Va transcripts and its absence in HAS1^{FL}, HAS1Vb and HAS1Vc transcripts obtained from the same patient suggests the presence of a new allelic variant of HAS1 in MM patients.

Although alternative splicing is a normal event contributing to protein diversity in humans, more than a dozen human cancers are associated with abnormalities in alternative splicing, particularly when intronic sequences are abnormally retained in the transcript. One cause of aberrant splicing is genetic variation (mutation and/or SNPs) in or near splice donor an/or acceptor sites and cis-splicing elements (exonic and intronic splicing enhancer and supressors (ESE, ESS), splicing branch point and polypyrimidine tracts within introns) as shown in cystic fibrosis (CFTR), breast cancer (BRAC1 and 2), and spinal muscular atrophy (SMA) (Ramalho, A. S., et al. J Med Genet 40:e88 (2003)), the consequences of which are exon skipping and/or intron retention in the transcript (Scholl, T. et al. Am J Med Genet 85:113 (1999); (Loo, J. C. et al. Oncogene 22:6387 (2003); (Brose, M. S. et al. Genet Test 8:133 (2004); Ketterling, R. P. et al. Hum Mutat 13:221 (1999); Neben, K. et al. Blood (2002); Krawczak, M. Hum Genet 90:41 (1992); Mayer K. et al. Biochim Biophys Acta 1502:495 (2000)). Aberrant HAS1 splice variants may promote malignant cell migration, enhance drug resistance and, as proposed below, may contribute to mitotic abnormalities and genetic instability in MM and WM.

HAS1 Splice Variant Proteins Synthesize HA.

Protein expression of all three HAS1 variants was shown by western blotting with polyclonal Ab raised against HAS1 peptides. In addition, using in silico methods, including the TMHMM Server v. 2.0, PSIPRED server, mGenTHREDER and MEMSAT (72-74), we evaluated the folding ability of HAS1 variants, and demonstrated that even though HAS1 variants are severely truncated proteins, they retain the ability to fold and preserve the Mg ion-binding pocket. Our work indicates that HAS1 and variants, HAS1Va and HAS1Vb, in combination with HAS3, are capable of synthesizing an extracellular HA matrix around MM CD19⁺ B cells. However normal B cells from healthy donors expressing only HAS3, and MM PC, which express HAS2 together with HAS3 but lack HAS1, are unable to synthesize extracellular HA as defined by the particle exclusion assay and HA staining. HAS1Va or HAS1Vb appear to be essential for synthesis of HA by malignant B cells. Only those patients having HAS1Vb expression were able to synthesize intracellular HA. Since the HAS1 variants appear to be absent from healthy cells, they may present valuable clinical targets for development of new therapeutics that are highly selective for malignant cells.

HASs in B lineage malignancy:

HASs have been shown to associate with malignant cell transformation (Zeng, C. et al. Int. J Cancer 77:396 (1998); Ichikawa, T. et al. J Invest Dermatol 113:935 (1999); Kosaski, R. et al. Cancer Res 59:1141 (1999); Itano, N. et al. Cancer Res 59:2499 (1999)) and an invasive phenotype. Competition by exogenous HA inhibits tumor growth (Herrera-Gayol, A. Exp Mol Pathol 72:179 (2002); Zeng, C. et al. Int J Cancer 77:396 (1998)). Of particular interest, since high dose dexamethasone is the most effective single drug treatment for multiple myeloma, glucocorticoids induce near total suppression of HAS1 and HAS2 (Stuhlmeir, K. M. et al. Rheumatology (Oxford) 43(2):261 (2004)).

When fibroblast-like synoviocytes were stimulated with TGF-beta, which is a potent activator of HAS1 mRNA transcription, treating them with hydrocortisone suppressed induced activation of HAS1 in a concentration- and time-dependent manner. Similar suppressive effects of hydrocortisone were observed when leucocytes isolated from synovial fluid of inflamed joints were used.

Our recent work shows cell-type specific expression for HAS-1 and HAS-2, while HAS-3 appears to be more ubiquitously expressed within the white blood cell types tested (Adamia, S. et al Blood 102:5211 (2003)). In MM, HAS1 and its aberrant splice variants are expressed exclusively by circulating malignant cells, while HAS2 is expressed only by bone marrow-localized/anchored malignant cells. In WM, single cell RT-PCR analysis of individual malignant B cells revealed that HAS1 full length (HAS1^{FL}) and the HAS1 splice variants are usually independently expressed, with frequent expression of aberrant HAS1 variants in the absence of transcript encoding the HAS1.

HASs, with the exception of HAS3, are all found in the blood cells of MM patients but not in the blood cells of healthy donors. In MM, among HAS isoenzymes, only HAS1 appears to synthesize extracellular HA, and only HAS1^{FL} and/or the HAS1 splice variants are associated with motile behavior (Adamia, S. et al. Blood in press (2005)). The expression of HAS1 and HAS1 variants by motile malignant B cells suggests that they are involved in oncogenic processes, particularly those contributing to the spread of MM. The expression of HAS1 and HAS1 variants, possibly in combination with HAS3, appears sufficient to synthesize the HA pericellular coat around MM B cells with a motile phenotype. Our longitudinal analysis shows that HAS1 and/or the HAS1 splice variants are usually expressed at the time of diagnosis, become sporadically undetectable during therapy, and reemerge prior to and during relapse (Adamia, S. et al Blood 102:5211 (2003)). In addition to the impact of HA in cancer cell migration/spread, and in mitosis, HA, and by extension HASs, may also play a role in the response of cancer cells to therapeutic drugs. It has been shown that HA oligomers are associated with drug resistance mechanisms of malignant cells (Misra, S. et al. J Biol. Chem. 278:25285 (2003)).

### SUMMARY OF THE INVENTION

The present art has suffered from a lack of simple genomic marker capable of identifying malignancies in cancer patients. As well, the art is in need of novel genetic markers for malignancies in general and predisposition to cancer.

In one aspect, the present invention provides for a method to detect presence of malignant cells in blood, bone marrow or other tissues comprising the detection of the presence of genetic mutations as disclosed herein in general and in Table 2 in particular.

In a further aspect the present invention provides for a method to determine whether malignant cells are present in patients with a premalignant condition comprising the detection of the presence of genetic mutations as disclosed herein in general and in Table 4 in particular.

In a further aspect present invention provides for a method to confirm diagnosis of malignancy comprising the detection of the presence of genetic mutations as disclosed herein in general and in Table 4 and Table 5 in particular.

In a further aspect the present invention provides for a method to distinguish between malignant and non malignant cells of the same morphologic or phenotypic type comprising separation of a single cell followed by detection of the presence of genetic mutations as disclosed herein in general and in Table 4 in particular.

In a further aspect the present invention provides a method to test individual cells for a malignant HAS genotype c comprising separation of a single cell followed by detection of the presence of genetic mutations as disclosed herein in general and in Table 4 and Table 5 in particular.

In another aspect the present invention provides for a method to determine the severity of disease prior to administration of chemotherapy or other cancer therapy course comprising the detection of the presence of genetic mutations as disclosed herein in general and in Table 4 and Table 5 in particular.

In another aspect the present invention provides for a method to predict whether aberrant HAS1 splicing is likely to occur, and whether targeted preventive therapy is warranted comprising the detection of the presence of genetic mutations as disclosed herein in general and in Table 4 and Table 5 in particular.

In another aspect the present invention provides for a novel therapeutic regimen for MM, WM and cancer comprising administration of a compound capable of interfering with, and preventing, aberrant RNA splicing resulting in aberrant HAS1 protein isoforms.

In another present the present invention provides for a method to detect malignant cells in an individual patient using patient-specific genomic marker(s) comprising isolation and identification of patient specific HAS1 genomic mutations followed by monitoring of the presence and quantity of HAS genomic mutations during and following disease treatment or therapy, said monitoring comprising the detection of the presence of genetic mutations as disclosed herein in Table 2 and Table 3

In another aspect the present invention provides for a method to determine the predisposition of a mammal to cancer or a proliferative disease or condition, comprising the detection of the presence of genetic mutations as disclosed herein, and in particular Table 5, Table 6, and Table 7. IN a preferred embodiment the mammal is human, and the cells examined for the presence of genetic mutations are buccal.

### BRIEF DESCRIPTION OF THE FIGURES

FIG. 1 shows the structure of HAS1Va, HAS1Vb and HAS1Vc genetic elements;

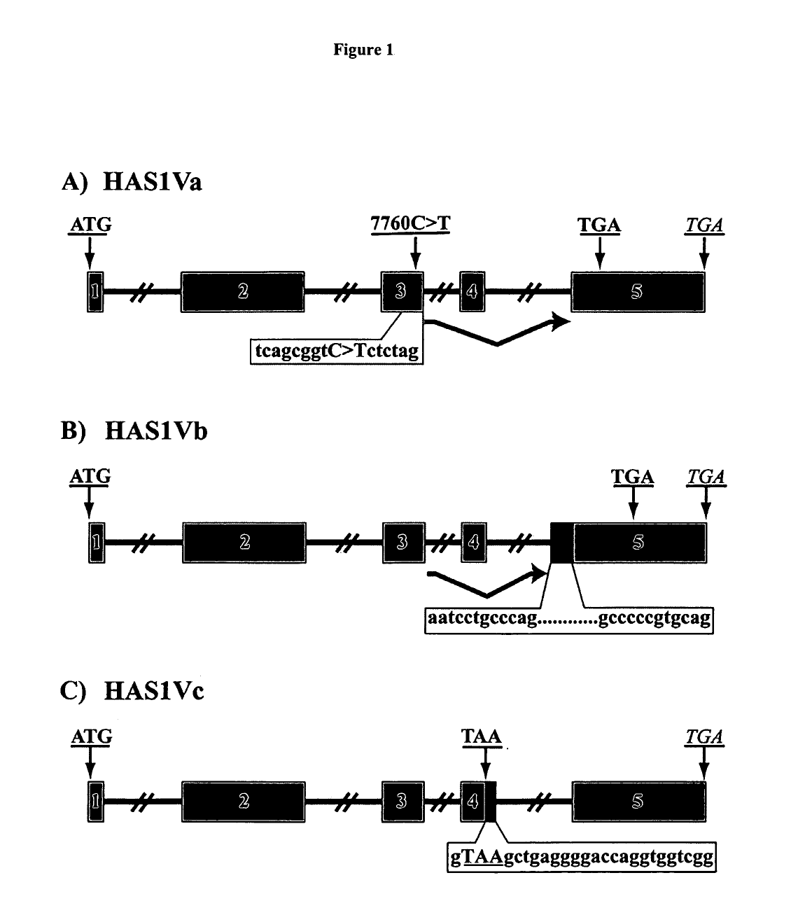

FIG. 2 shows the structure and function of HAS proteins;

FIG. 3 shows HAS1 genomic sequence with unique, recurrent and known genetic variations;

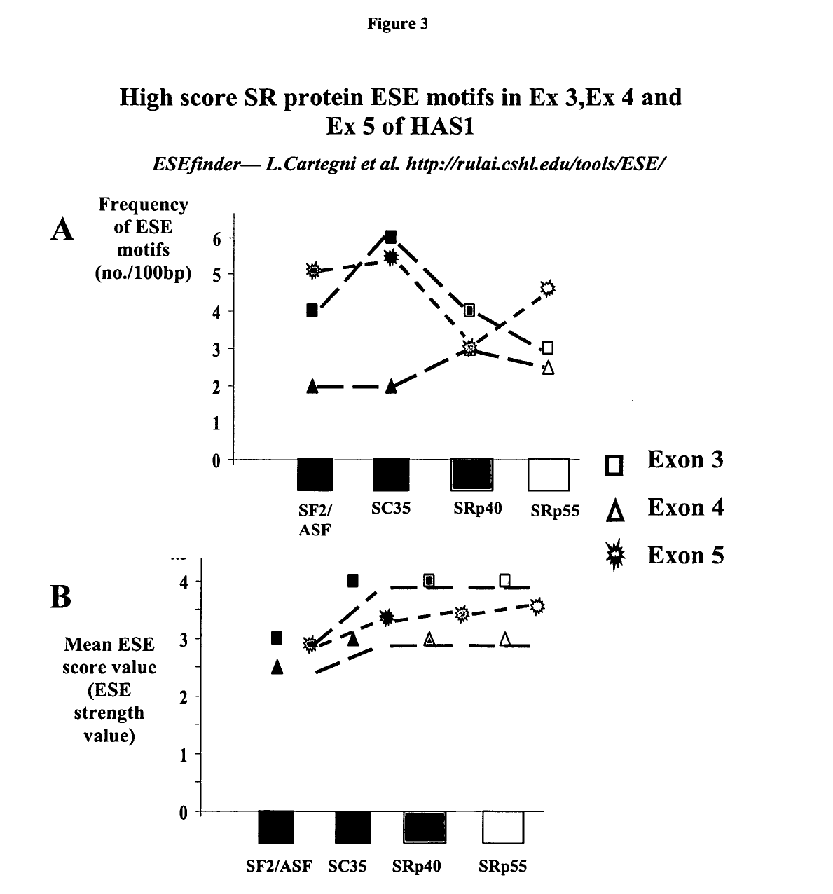

FIG. 4 shows ESE/ISE and ESS/ISS affected by genetic variations identified on HAS1 gene;

FIG. 5 shows Secondary structure of HAS1 gene before and after genetic variations identified on exons and introns; and

FIG. 6 shows a schematic diagram of the effect of genetic variations on gene transcription;

### DETAILED DESCRIPTION OF THE PRESENT INVENTION

Definitions

As used herein “stringent conditions” means conditions that detect a nucleic acid molecule with at least 90%, preferably at least 95%, nucleotide sequence homology to the probe or primer sequence. See Sambrook et al. Molecular Cloning a Laboratory Manual Cold Spring Harbor Press 2 ed, (1989); PCR Primer: A Laboratorv Manual. Carl Dieffenbach Ed. Cold Spring Harbor Press (1995), for a selection of conditions suitable for washing and hybridizing nucleic acids allowing for stable and specific duplex formation and/or Reverse Transcriptase Polymerase Chain Reaction (RT-PCR). Stringent conditions are those that either employ low ionic strength and high temperature for washing, or employ a denaturing agent during hybridization.

As used herein “Polymerase Chain Reaction” or “PCR” refers to the process or technique of increasing the concentration of a segment of a target sequence of pre-selected genomic material comprised of, but not limited to, DNA, mRNA, cDNA, or fragments thereof, as generally described in U.S. Pat. Nos. 4,683,195, 4,683,202, and 4,965,188.

As used herein “isoenzyme variants” refers to a protein resulting from the alteration of the native HAS1 enzyme arising from post-translational or pre-translational modification.

As used herein “disease” means a state in a mammal which may directly or indirectly lead to a cellular, cell population, or systemic state detrimental to the mammal.

As used herein, the term “probe” refers to an oligonucleotide, single-stranded or double-stranded, produced synthetically or occurring naturally; that is capable of selectively binding to a nucleic acid of interest.

As used herein, the term “primer” refers to an oligonucleotide produced synthetically or naturally occurring, which is capable of acting as a point of initiation of nucleotide synthesis when placed under conditions in which nucleotide synthesis extending from the primer, complimentary to a nucleic acid strand, is possible.

As used herein, “therapeutic” refers to a method or process to vary the expression or transcription of HAS 1 or HAS 1 isoenzyme variants in a cell or cell population; in which the expression, transcription or post-translational modification of HAS1 or HAS1 isoenzyme variants, or lack thereof, is deleterious to the cell or cell population or gives rise to a susceptibility to a condition which is deleterious to the cell or cell population.

As used herein, “microfluidic devices”, sometimes termed “lab on a chip”, “microfluidic chips” or “microsystem platforms” refer to the result of applying microelectronic fabrication technologies to produce a network of wells and channels etched into glass and/or molded into polymers that are bonded to glass or silicon chips. Within these wells and microchannels, cells and reagents can be manipulated by a variety of methods including gravity feed, applying electric or magnetic fields and results detected by, for example, image analysis or optical means. Microfluidic chips provide for PCR reactions and analysis of PCR products (Footz, T. S. et al. Electrophoresis 22:3868 (2001); Obeid, P. J. et al. Analytical Chemistry 75:288 (2003); Backhouse C. J. et al. Electrophoresis 24:1777 (2003)). They enable high resolution separations through polymer-filled microchannels using capillary electrophoresis of e.g. multiple PCR products, and can exhibit a high level of integration by combining multiple functions on a single chip, for example cell sorting and RT-PCR reactions for gene expression or genomic profiles of a given cell or population of cells (Backhouse, C. J. et al. Proceedings of the International Conference on MEMS, NANO and Smart Systems 377 (2003)). Within a microfluidic device, sample processing can be implemented and cells can be separated by a variety of means, including dielectrophoresis, and processed in a variety of ways, including analysis of HAS gene expression as shown here. In the future, microsystem platforms incorporating microfluidics chip-based sample processing and analysis may replace more conventional methodologies for applications such as genotyping.

HAS Abnormalities in Other Cancers

HAS1 is a prognostic factor in MM, ovarian and colon cancer (Adamia, S. et al. Blood 102:5211 (2003); Yamada, Y. et al. Clin. Exp. Metastasis 21:57 (2004); Yabushita, H. Oncol. Rep. 12:739 (2004)). Although as yet there have been no reports of HAS1 splice variant expression in ovarian and colon cancer, based on observations in MM and WM, this would be predicted by one skilled in the art. Overexpressed HAS2 and HAS3 have been identified in prostate cancer (Tsuchiya, N. et al. Am J Pathol 160:1799 (2002); Liu, N. et al. Cancer Res 61:5207 (2001); (Simpson, M. A. et al. J Biol Chem. 276: 17949 (2001)). HAS2 and HAS3 are overexpressed in malignant mesothelioma (Liu, Z. et al. Anticancer Res 24:599 and HAS3 is overexpressed in glioma (Enegd, B. Neurosurgery 50:1311 (2002)). As well, HAS 1 variants are observed to correlate with production of extracelluar HA (Adamia, S. et al. Blood 105:4836 (2005)) (Table 1).

TABLE 1
HAS 1 variants correlate with HA production by MM B cells.

| Number of | | |
|---|---|---|
| patients | HAS RNA expression pattern | HA production |
| 2 | HAS1Va, HAS3 | Extracellular HA matrix |
| 3 | HAS1Va, HAS1, HAS3 | Extracellular HA matrix |
| 3 | HAS1Va, HAS1Vb, HAS1, | Extracellular HA matrix & |
| | HAS3 | intracellular HA |
| 2 | HAS1Vb, HAS1, HAS3 | Intracellular HA & very |
| | | weak extracellular HA |
| 3 | HAS1, HAS3 | No HA production |
| 2 | HAS3 | No HA production |

HAS1Vb Predicts for Poor Survival in Multiple Myeloma.

In MM, the presence of HAS isoenzyme variants in the blood correlates with poor survival (Adamia, S. et al. Blood 102:5211 (2003)), but to date no significant correlations have been detected for HAS isoenzyme variants expressed by bone marrow-localized malignant cells. This suggests that HAS isoenzyme variants are upregulated in the blood-borne components of the myeloma clone and are biologically relevant markers of circulating tumor burden. A highly significant correlation between poor survival and expression of HAS genes in blood borne cells is found for the intronic splice variant HAS1Vb, and a strong trend towards clinical correlations with poor outcome is seen for HAS1-FL and HAS1Va (U.S. Pat Application No. 20050003368). The strong association between HAS1Vb and survival, taken together with the rare detection of HAS1Vb in the bone marrow, suggests that HAS1IVb may be preferentially upregulated in circulating malignant cells. Analysis of purified B lineage subsets from MM and WM patients confirms this. HAS1Vb is expressed by circulating B cells as identified by their phenotypic marker profiles, but is not detected in BM-localized B or plasma cells (Adamia, S. et al. Blood 102:5211 (2003)). HAS1 thus represents a new type of prognostic marker that reflects biologically important properties of a malignant clone as it undergoes stepwise oncogenesis and/or disease progression.

HAS1 and Genetic Instability

HAS1 gene expression may promote genetic instability. This idea is supported by the observation that circulating clonal B cells in myeloma patients are extensively DNA aneuploid with, on average, 1.07 excess DNA content, equivalent to an additional 3.2 chromosomes. This provides evidence for genetic instability in the malignant MM B cells that overexpress HAS1 and its variants. Regardless of mechanism, the significant correlation between poor survival and the expression of HAS1 and its splice variants by circulating B cells suggests a key role for expression of HASs by “stem cell” components of the MM clone that circulate in the blood and mediate malignant spread to distant bone marrow sites. The detection of novel HAS1 variants at high levels in MM B cells and their absence from normal B cells, as well as from other cell types, suggests that aberrant HAS1 splicing is characteristic of malignant cells. The detection of HAS1 variants in monoclonal gammopathies of undetermined significance (MGUS) suggests that their expression may be an early event in the genesis of MM.

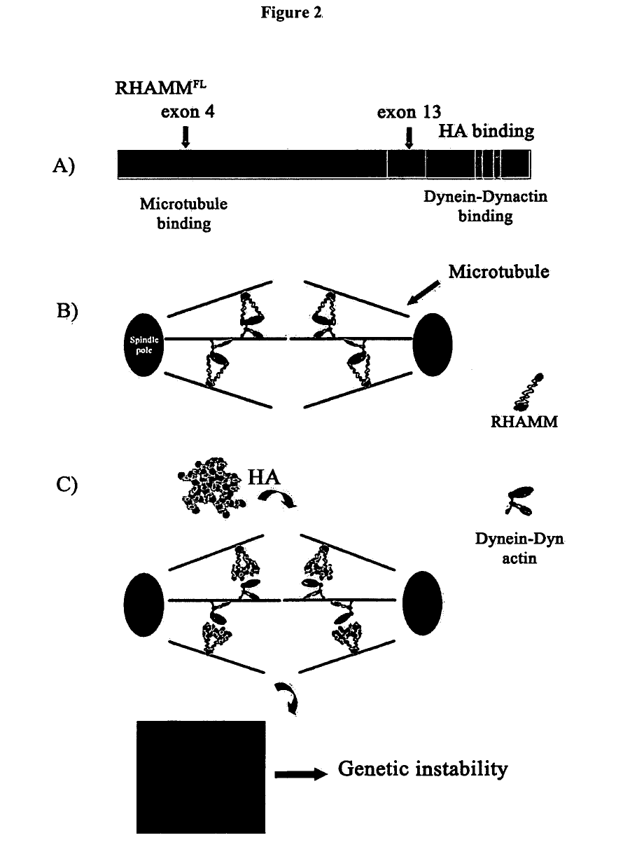

The enzymatically active part of full length HAS1 protein is intracellular. Based on their sequences and predicted tertiary structures, we speculate that HAS1 variants are intracellular and/or membrane-anchored isoenzymes retaining enzymatically active domains that are likely to synthesize intracellular HA (FIG. 2), a ligand for intracellular RHAMM, thereby contributing to the RHAMM-induced dysregulation of mitosis and subsequent chromosomal abnormalities. The evidence disclosed herein, coupled with our previous observations that treatment with HA triggers redistribution of intracellular RHAMM to the surface, leads to the conclusion that aberrant HAS1 is a key regulator of RHAMM redistribution and that together, HAS1 and RHAMM contribute to the generation of increasingly aggressive clones in MM and WM. Additionally, intracellular HA in concert with RHAMM may contribute to RHAMM-microtubule interaction.

Influence on Splicing of Genetic Variation in Genomic DNA

As indicated above, HAS gene expression analysis has demonstrated abnormalities of HAS1 in MM and WM patients. (Adamia, S. et al. Semin Oncol 30:165 (2003); Adamia, S. et al. Blood 105:4836 (2005)). The expression patterns of HAS1 and splice variants in MM and WM patients are likely to occur in other cancers characterized by abnormalities in HASs. HAS1Va (HAS1T) is the result of exon skipping which causes a frame shift (Adamia, S. et al. Semin Oncol 30:165 (2003); Adamia, S. et al. Blood 105:4836 (2005)). However, HAS1Vb and HAS1Vc are the result of intronic splicing, since both of these transcripts retain part of intron 4 either at the 3′ splice site of alternative exon 4 or at the 5′ splice site of exon 5 (Adamia, S. et al. Blood 105:4836 (2005)). These splicing aberrations generate premature stop codons on spliced HAS1 transcripts leading to severe truncation of the encoded proteins. Using bioinformatics analysis in combination with western blotting performed on lysates obtained from MM cell lines, it has been verified that the aberrantly spliced HAS1 transcripts encode proteins which are able to fold properly and produce extracellular and/or intracellular HA. Production of HA has been demonstrated by particle exclusion assay and HA staining. (Adamia, S. et al. Blood 105:4836 (2005)).

Cancer results from aberrations in gene expression and aberrant splicing is a major regulator of gene expression (Adamia, S. et al. Blood 105:4836 (2005); Hastings, M. L. et al Curr Opin Cell Biol 13:302 (2001); Bartel, F. et al. Cancer Cell 2:9 (2002)). Pre-mRNA processing, which occurs in the nucleus of the cell, is a complex process that includes pre-mRNA splicing (Hastings, M. L. et al. Curr Opin Cell Biol 13:302 (2001)). Splicing of a given gene requires activation of more than 100 proteins, including splicing factors and at least 5 small nuclear RNA protein particles (Caballero, O. L. et al. Dis Markers 17:67 (2001)).

Specificity of splicing is known to be defined by the 5′ and 3′ splicing sites and branch points (Nissim-Rafinia, M. et al. Trends Genet 18:123 (2002); Caballero, O. L. et al. Dis Markers 17:67 (2001); Caceres J. F. et al. Trends Genet 18:186 (2002)). However, evidence reported in the literature suggests the importance of other cis-splicing elements, such as exonic splicing enhancers (ESE) and exonic splicing suppressors (ESS) and their intronic counterparts (ISE and ISS), and polypyrimidine tracts. (Caballero O. L. et al. Dis Markers 17:67 (2001); Nissim-Rafinia, M. et al. Trends Genet 18:123 (2002)). Furthermore, mutations occurring in ESE/ISE and ESS/ISS in combination with an aberrant expression of splicing factors (SF) play a significant role in aberrant splicing (Caballero, O. L. et al. Dis Markers 17:67 (2001); Caceres, J. F. et al. Trends Genet 18:186 (2002); Nissim-Rafinia, M. et al. Trends Genet 18:123 (2002)). However, additional mutations on polypyrimidine tracts and on the splicing branch points are required to activate cryptic splice sites and achieve aberrant splicing (Caballero, O. L. et al. Dis Markers 17:67 (2001); Dominski, Z. et al. Mol Cell Biol 11:6075 (1991); Chabot, B. et al. Mol Cell Biol 17:1776 (1997); Cote, J. et al. RNA 3:1248 (1997)).

The current art suggests that high specificity of splice site identification by the splicing machinery can not be fully explained by primary sequence conservation. During splicing, introns fold into secondary structure to localize splicing branch-point at the optimal distance from 5′ splicing site and facilitate assembly of splicing complexes. Alteration of the secondary structure of pre-mRNA, which can be induced by mutations, compromise the splicing pattern of a gene (Buratti et al. Mol Cell Biol 24:10505 (2004)).

To evaluate the factors leading to aberrant HAS1 splicing, ESEs located within the alternatively spliced exon 4 and in the adjacent exon 3 were identified. In addition, the NCBI database was screened to identify mutations and/or single nucleotide polymorphisms (SNPs) on the alternative exon 4 and on exon 3. No mutations were found on alternative exon 4. However, the HAS1 833A/G SNP is located on exon 3. Genetic variation of the 833A/G SNP in exon 3 of HAS1 (Ch19q13.4) in patients was determined using the Taqman SNP Genotyping assay. 86.8% of patients with WM (79/91 tested, p=0.0004) and 85% of MM (230/270 tested, p=0.000002) are homozygous for HAS1 833G/G, as compared to 65% of healthy donors (81/124 tested). No healthy donors or patients have yet been found with homozygous HAS 1 833A/A, suggesting this may be lethal. Homozygosity in WM and MM is statistically significant as measured using three different tests, for WM (p=0.0004 as compared to healthy donors) and for MM (p=0.000002 as compared to healthy donors). HAS1 833A/G homozygosity reflects the germline constitution of the patient, suggesting it may be a predisposing factor for paraproteinemias, perhaps by influencing HAS1 splicing events as discussed below. The HAS1 833A/A genotype was not detected in any patient or healthy donors screened to date, and is presumptively lethal. The same group of WM patients was screened for expression of HAS1 transcripts and splice variants. It was found that increased homozygosity in locus Ch19q13.4 correlated with expression of aberrant splice variants of HAS1, particularly intronic HAS1Vb and HAS1Vc. Only WM patients with HAS1 833G/G genotype expressed either HAS1Vb and/or HAS1Vc with or without full length HAS1. Therefore aberrant splicing of the HAS1 gene in MM and WM patients may be related to the presence of the HAS1 833G/G genotype.

To investigate effects of HAS1 833A/G SNP on HAS1 aberrant splicing ESEs were identified, considering that they are present in constitutive and alternative exons and are required for efficient splicing. Exonic splicing enhancers are recognized by serine/arginine-rich (SR) proteins essential for alternative splicing (Blanchette, M. et al. RNA 3: 405 (1997); Blencowe, B. J. Trends Biochem Sci 25:106 (2000); Zahler A. M. et al. Mol Cell Biol 13:4023 (1993); Zahler A. M. et al. Science 260:219 (1993); Blencowe, B. J. Trends Biochem Sci 25:106 (2000)). A computational approach was used, termed an in silico method, with ESE finder (http://rulai.cshl.edu/tools/ESE/) and exons 3 and 4 were analyzed using SF2/ASF, SC35, SRp40 and SRp55 motif-scoring matrices, derived from pools of the functional enhancer sequences selected from the literature (Cartegni, L. et al. Nucleic Acids Res 31:3568 (2003); Cartegni,l L. et al. Nat Struct Biol 10:120 (2003)). Threshold settings for the analysis were 1.956 for SF2/ASF, 2.383 for SC35, 2.670 for SRp40 and 2.676 for SRp55.

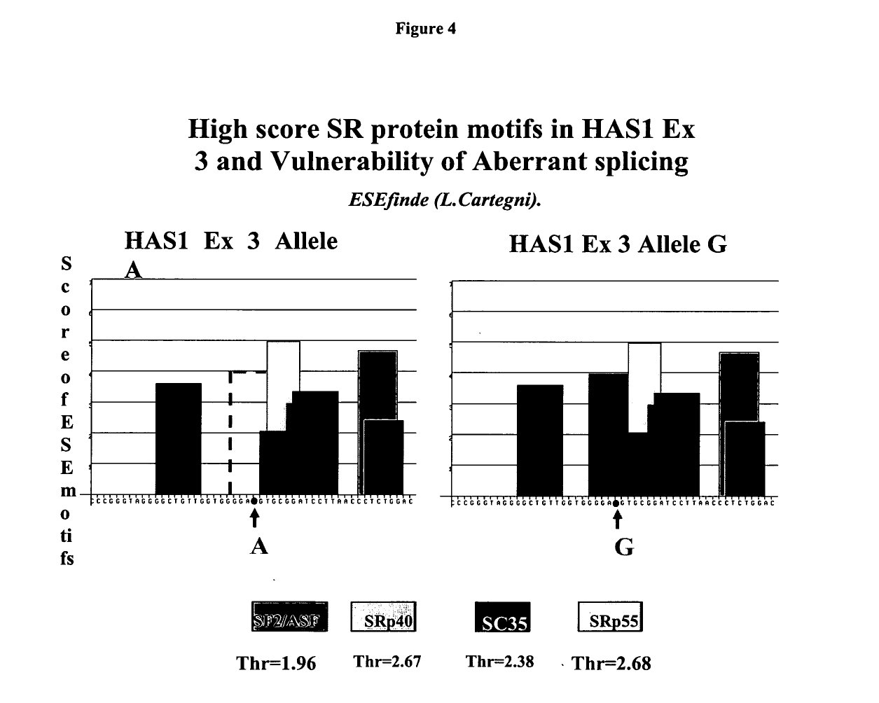

The frequency of the sequence motifs which attract the indicated SF and are most highly expressed in any given human cell nucleus were identified. Screening of both exon 3 and alternative exon 4 of the HAS1 gene demonstrated that the frequency of sequence motifs, which attract the designated SFs, is higher on exon 3 than on alternative exon 4 and exon 5 (FIG. 2A). In addition, using ESE finder the affinity of these SFs to the sequence motifs distributed on exons 3, 4, and 5 were identified (FIG. 2b). This analysis also demonstrated that the binding affinity of SFs is higher for exon 3 than for exon 4 and 5, and binding affinity of these SF are high for exon 5 than for exon 4, suggesting an enhanced role for exon 3 in skipping of exon 4 and in aberrant splicing. A similar phenomenon has been described by Steiner et al for the aberrant splicing of CFTR gene transcripts (Steiner, B. et al. Hum Mutat 24:120 (2004)). Based on data obtained from ESE analysis putative ESEs were predicted and mapped the HAS1 833A/G SNP with an ESE. As FIG. 4 shows, for HAS1 833A, the mutation/SNP abolishes the putative ESE. This may explain why HAS1 833A/A genotype is so far undetected in MM and WM patients or healthy donors.

The HAS1 833A/A mutation may disrupt the splicing mechanism, thereby rendering the HAS1 833A/A a lethal genotype. However, for HAS1 833G, the putative ESE remains intact, with the calculated affinity of SRp55 to ESE located on exon 3 being higher than that for SF2, in addition to higher binding affinity of all analyzed SF on exon 3. The HAS1 833G/G genotype may create a “gene dosage” effect in the nucleus of malignant WM cells, perhaps leading to the activation of distal 3′ splicing site and causing exon 4 skipping (Longman, D. et al. Curr Biol 11:1923 (2001); Ring, H. Z. et al. Mol Cell Biol 14:7499 (1994)). However, the population of healthy donors also includes individuals with a HAS1 833G/G genotype who lack expression of HAS1 or its variants. Thus, the increased affinity of SFs for ESE having HAS1 833G appears to be necessary but not sufficient to activate cryptic splice sites in HAS1. Thus the HAS1 833G/G genotype predisposes to WM and MM, and thereby serves as a diagnostic indicator.

Though an exact understanding of the mechanism is not necessary to practise the present invention, based on the evidence described herein, the high affinity of SRp55 is proposed to aggregate specific splicing proteins, thus promoting the skipping of short, alternative exon 4. It is propsed that additional mutations are required to mediate aberrant splicing of this gene (Steiner, B. et al. Hum Mutat 24:120 (2004); Liu, H. X. et al. Nat Genet 27:55 (2001)). This explains why healthy donors with HAS1 833G/G genotype do not express aberrant splice variants of HAS1. The HAS1 833G/G appears insufficient to promote aberrant splicing of this gene. The present invention provides for the HAS1 833G/G genotype as indicating a predisposition of a patient to MM, WM and by correlation other cancers characterized by abnormalities in HASs. Bioinformatics analysis provides that HAS1 833G/G genotype, in combination with additional mutations could activate cryptic splice sites of the HAS1 gene and promote aberrant HAS1 gene splicing. Therefore the HAS1 833G/G genotype is a predictive marker of cancer. In particular, the best mode of the present invention discloses predictors of cancer based in genomic DNA rather than in cDNA or RNA as has been disclosed in the art previously, in particular HAS1 833A/A, HAS1 833 G/G, those listed in Tables 2 and 3; more particularly those listed in Tables 4, 5, 6 and 7 and more particularly in Tables 6 and 7.

Sequencing of genomic DNA from exon 3, intron 3, exon 4, intron 4 and exon 5 of the HAS1 gene.

Exons 3 and 4 and introns 3 and 4, from five WM patients and six MM patients and identified genetic variations not previously known to the art were identified. The genetic variants of HAS1 disclosed herein and those previously reported SNPs, have been mapped to define HAS1 haplotypes, based on their proximity to splice sites and cis splicing elements (ESE/ISE and ESS/ISS) that are important for correct splice-site identification and are distinct from classical splicing signals. These elements can act both as an enhancers or silencers of splicing. In particular, exonic splicing enhancers (ESEs) are prevalent. ESE have been identified using on-line tools ESEfinder release 2 (based on SF2/ASF,SC35, SRp40 and SRp55 motif-scoring matrices), RESCUE-ESE and RESCUE-ISE (httn://genes.mit.edu/burgelab/rescue-ese) Web Server. Using this approach, for the cancer cells from eleven patients, multiple types of genetic variations in HAS1 have been identified and disclosed herein. These include point mutations, nucleotide(s) insertions and deletions, tranversions and transitions. Together these are referred to as genetic variations of a given type, to be inclusive of all categories of variation described above (mutations, insertions and deletions).

Furthermore, based on the distribution of these genetic variations (GV) in HAS1 genomic DNA, four broad categories have been identified:

1) variations that have been previously reported in online databases;

2) variations that are unique to the tumor clone in individual patients;

3) variations that are disease restricted (e.g. recurrent only in MM or only in WM patients); and

4) variations that are recurrent in all WM and MM individuals tested—that is they are present in the HAS1 genomic DNA of the cancer cells from all 11 patients studied.

The present invention discloses novel variations comprising types 2-4 and the detection of variations in a patient comprising types 1-4 as a diagnostic for the existence of cancer or proliferative disease or disorder, in particular MM or WM; and as a diagnostic for a predisposition to cancer or proliferative disease or disorder, in particular MM or WM. As known in the art, genomic variations (including SNPs) can influence spliceosome assembly and thus may contribute to aberrant splicing of HAS1 in cancer patients. Though an exact understanding of the mechanism is not needed to practise the present invention, it is proposed that aberrant splicing of HAS1 results from activation of cryptic splice sites, which lead to exon skipping and/or intron retention. In turn, activation of cryptic donor and/or acceptor splice sites can be promoted by the mutations occurring on ESE/ISE, ESS/ISS and/or at the splicing branch point and polypyrimidine tracts.

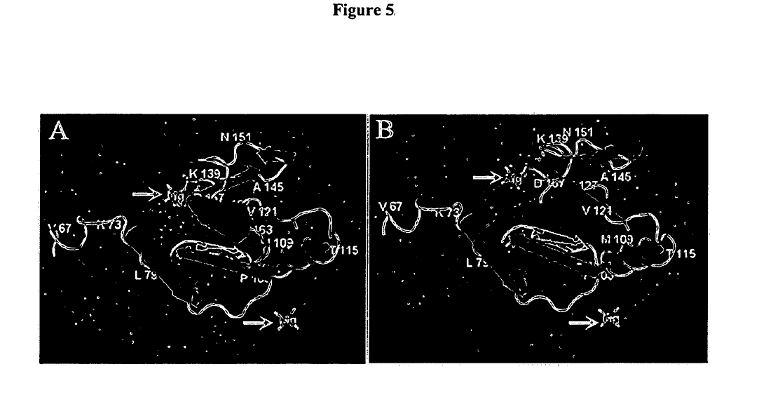

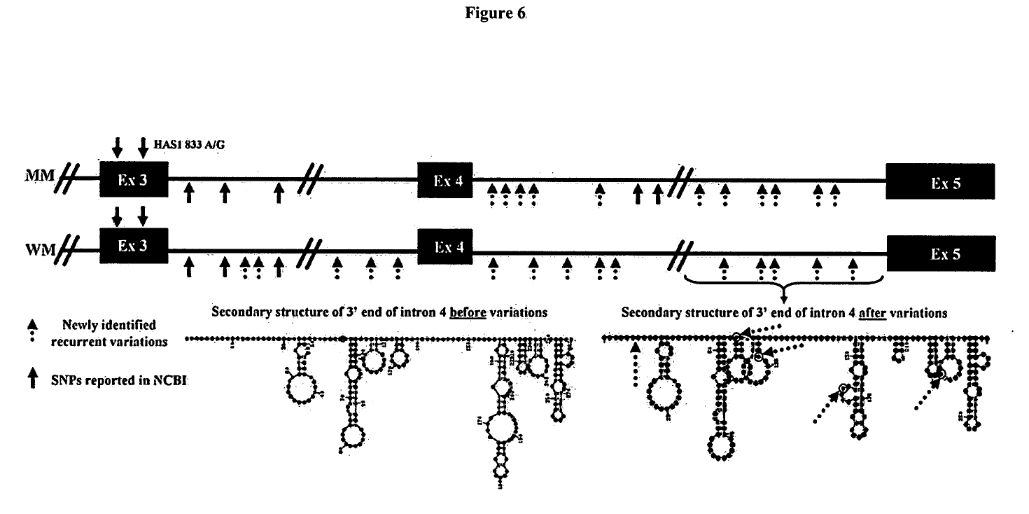

The exons and introns were sequenced from 11 different patients (6 with MM and 5 with WM) to identify novel SNPs and identify whether or not recurrent genetic variations of HAS1 are detectable in malignant B cells. Sequencing has been comprehensive, with 3-5 subclones sequenced both directions for each exon or intron of each patient. Genetic variations were identified as already in the NCBI SNP database or as novel variations. Although we identified novel variations that were unique to individual patients, we were surprised to find that many of the genetic variations in exons and introns of HAS1 were recurrent for all malignant clones analyzed (from 11 different patients). These newly identified recurrent variations are indicated in FIG. 4 (dotted arrows). FIG. 5 shows the proposed changes to the HAS1 gene. FIG. 6 shows hypothized alteration of secondary structure imposed by the genetic variations in intron 4.

As can be seen in Table 2, and summarized in Tables 4, 5, 6 and 7; a number of MM in general, and patient specific, genetic variations are observed to occur. As well, as can be seen in Table 3, and summarized in Tables 4, 5, 6 and 7, a number of WM in general and patient specific genetic variations are observed to occur. As will be detailed below, the genetic variations in MM and WM fall into three categories based on the cell types in which they are detected, in any given patient—those that are present in the tumor (tumor specific), those that are present in the hematopoietic lineage (hematopoietic lineage), and those that are present in all cells of the body (germline origin). Genetic variations of germline origin include both novel SNPs first identified here and SNPs that have been previously reported in the art but whose clinical value has not been previously established as predictive markers for disease. For all categories of genetic variation, their use as markers for predicting disease susceptibility, as early indicators of disease stage or for monitoring frank malignancy provides different types of clinically valuable information, as described below.

TABLE 2
Recurrent and unique mutations in exons 3-4 and introns 3-4 of gHAS1 from
Multiple Myeloma patients

| | | | | | | |
|---|---|---|---|---|---|---|
| Types of GV | GV | NT | Seq NT | Frequency | Comments | Effects |

Exon 3

| | | | | | | |
|---|---|---|---|---|---|---|
| Transition | g > A | 24488419 | 3978 | Unique | Germline origin | Gly > Gly |
| Transition | t > C | 24488429 | 3988 | recurrent | novel SNP | Cys > Arg |
| Transversion | t > A | 24488429 | 3988 | Unique | Tumor specific | Cys > Ser |
| Transition | t > C | 24488434 | 3993 | recurrent | Hematopoietic linage | Val > Ala |
| Transition | t > C | 24488436 | 3995 | Unique | Tumor specific | Cys > Cys |
| Transition | g > A | 24488455 | 4014 | Unique | Hematopoietic linage | Cys > Tyr |
| Transition | t > C | 24488457 | 4016 | recurrent | Hematopoietic linage | Ala > Ala |
| Transition | c > T | 24488458 | 4017 | Unique | Germline origin | Ala > Val |
| Transition | t > C | 24488467 | 4026 | Unique | Hematopoietic linage | Val > Ala |
| Transition | c > T | 24488505 | 4064 | Unique | Tumor specific | Ser > Ser |
| Transition | c > T | 24488517 | 4076 | Unique | Tumor specific | Asp > Asp |
| Transition | c > T | 24488541 | 4100 | recurrent | SNP-NCBI rs 11084111 | Asp > Asp |
| Transition | t > C | 24488553 | 4112 | Unique | Tumor specific | Ala > Ala |
| Transition | c > T | 24488564 | 4123 | Unique | Tumor specific | Arg > Trp |
| Transition | a > G | 24488575 | 4134 | Unique | Hematopoietic linage | Asp > Gly |
| Transition | c > T | 24488608 | 4167 | Unique | Tumor specific | Pro > Leu |

Intron 3

| | | | | | |
|---|---|---|---|---|---|
| Transversion | t > A | 24488128 | 3687 | recurrent | SNP-NCBI rs 11669079 |
| Transition | t > C | 24488141 | 3700 | unique | Tumor specific |
| Transition | a > G | 24488147 | 3706 | unique | Germline origin |
| Transition | g > A | 24488191 | 3750 | unique | Hematopoietic linage |
| Transition | g > A | 24488209 | 3768 | recurrent | SNP-NCBI rs 11084109 |
| Transition | g > A | 24488249 | 3808 | unique | Hematopoietic linage |
| Transition | g > A | 24488267 | 3826 | Unique | SNP-NCBI rs 11084110 |
| Transition | g > A | 24488303 | 3862 | unique | Hematopoietic linage |
| Transition | a > G | 24488320 | 3879 | Unique | Tumor specific |
| Transition | a > G | 24488361 | 3920 | recurrent | Hematopoietic linage |
| Transition | g > a | 24488374 | 3933 | Unique | Hematopoietic linage |
| Transversion | g > T | 24488395 | 3954 | Unique | Tumor specific |
| Transition | c > T | 24488405 | 3964 | Unique | Germline origin |
| Transversion | a > T | 24488408 | 3967 | Unique | Tumor specific |

Exon 4

| | | | | | | |
|---|---|---|---|---|---|---|
| Transversion | a > T | 24487724 | 3283 | Unique | Hematopoietic linage | Met > Leu |
| Transversion | g > C | 24487726 | 3285 | Unique | Hematopoietic linage | Arg > Pro |

Intron 4

| | | | | | |
|---|---|---|---|---|---|
| Transition | a > G | 24485564 | 1123 | Unique | Tumor specific |
| Transition | t > C | 24485574 | 1133 | Unique | Tumor specific |
| Transition | a > G | 24485594 | 1153 | Unique | Tumor specific |
| Transition | c > T | 24485595 | 1154 | recurrent | Novel SNP |
| Transition | g > A | 24485621 | 1180 | unique | Germline origin |
| Transition | a > G | 24485658 | 1217 | Unique | Tumor specific |
| Transition | t > C | 24485663 | 1222 | Unique | Tumor specific |
| Deletion | del C | 24485686 | 1245 | Unique | Tumor specific |
| Transition | a > G | 24485727 | 1286 | Unique | Tumor specific |
| Transition | t > C | 24485767 | 1326 | Unique | Hematopoietic linage |
| Transversion | g > T | 24485780 | 1339 | unique | Hematopoietic linage |
| Transition | c > T | 24485793 | 1352 | recurrent | Novel SNP |
| Transition | t > C | 24485797 | 1356 | Unique | Tumor specific |
| Transition | t > C | 24485801 | 1360 | recurrent | Hematopoietic linage |
| Transition | g > A | 24485802 | 1361 | Unique | Tumor specific |
| Transition | g > A | 24485805 | 1364 | Unique | Tumor specific |
| Insertion | x TTTA | 24485814 | 1373 | Unique | Germline origin |
| Deletion | x TTTA | 24485817 | 1376 | recurrent | Germline origin |
| Transition | g > A | 24485844 | 1403 | Unique | Tumor specific |
| Transition | t > C | 24485899 | 1458 | Unique | Tumor specific |
| Transversion | t > G | 24485936 | 1495 | Unique | Tumor specific |
| Deletion | del C | 24485951 | 1510 | Unique | Tumor specific |
| Insertion | inst (T)s | 24485967 | 1526 | recurrent | Germline origin |
| Deletion | del T | 24485969 | 1528 | recurrent | Tumor specific |
| Transition | a > G | 24486002 | 1561 | Unique | Tumor specific |
| Transition | a > G | 24486015 | 1574 | Unique | Tumor specific |
| Transition | t > C | 24486030 | 1589 | Unique | Hematopoietic linage |
| Transition | t > c | 24486116 | 1675 | Unique | Hematopoietic linage |
| Transition | c > T | 24486141 | 1700 | recurrent | SNP-NCBI rs 8104157 |
| Insertion | inst (Ts) | 24486140 | 1699 | recurrent | Germline origin |
| Insertion | inst (Ts) | 24486142 | 1701 | recurrent | Germline origin |
| Transition | t > C | 24486244 | 1803 | Unique | Tumor specific |
| Transversion | g > T | 24486419 | 1978 | Unique | Hematopoietic linage |
| Transition | a > G | 24486494 | 2053 | Unique | Hematopoietic linage |
| Transversion | c > G | 24486532 | 2091 | recurrent | SNP-NCBI rs 4802848 |
| Transversion | c > A | 24486533 | 2092 | recurrent | SNP-NCBI rs 4802849 |
| Transversion | a > C | 24486576 | 2135 | Unique | Hematopoietic linage |
| Transition | a > G | 24486597 | 2156 | recurrent | novel SNP |
| Transition | a > G | 24486671 | 2230 | unique | Germline origin |
| Transition | t > C | 24486744 | 2303 | unique | Hematopoietic linage |
| Transition | t > C | 24486747 | 2306 | Unique | Tumor specific |
| Transversion | g > T | 24486773 | 2332 | Unique | Tumor specific |
| Transition | a > G | 24486788 | 2347 | Unique | Hematopoietic linage |
| Transition | a > G | 24486815 | 2374 | Unique | Tumor specific |
| Transition | t > C | 24486829 | 2388 | Unique | Hematopoietic linage |
| Transversion | g > C | 24486871 | 2430 | recurrent | SNP-NCBI rs 4802850 |
| Transition | a > G | 24486943 | 2502 | Unique | Tumor specific |
| Transition | a > G | 24487004 | 2563 | Unique | Tumor specific |
| Transition | g > A | 24487010 | 2569 | unique | Germline origin |
| Transition | g > A | 24487089 | 2648 | recurrent | SNP-NCBI rs 7254072 |
| Transversion | t > G | 24487116 | 2675 | recurrent | SNP-NCBI rs 11667949 |
| Transition | g > A | 24487120 | 2679 | Unique | Hematopoietic linage |
| Transition | t > C | 24487126 | 2685 | Unique | Tumor specific |
| Transition | t > C | 24487140 | 2699 | Unique | Tumor specific |
| Transition | t > C | 24487156 | 2715 | Unique | Tumor specific |
| Transversion | a > C | 24487185 | 2744 | Unique | Tumor specific |
| Transition | c > T | 24487188 | 2747 | Unique | Tumor specific |
| Transition | a > G | 24487214 | 2773 | Unique | Tumor specific |
| Transition | t > C | 24487227 | 2786 | unique | Germline origin |
| Transition | g > A | 24487278 | 2837 | unique | Hematopoietic linage |
| Transition | c > T | 24487283 | 2842 | Unique | Tumor specific |
| Transversion | g > C | 24487148 | 2707 | recurrent | SNP-NCBI rs 11667974 |
| Transition | t > C | 24487459 | 3018 | unique | Germline origin |
| Transition | a > G | 24487493 | 3052 | unique | Tumor specific |
| Transition | g > A | 24487556 | 3115 | Unique | Hematopoietic linage |
| Transition | a > G | 24487577 | 3136 | unique | Germline origin |
| Transition | t > C | 24487579 | 3138 | unique | Hematopoietic linage |
| Transition | t > C | 24487588 | 3147 | Unique | Germline origin |
| Deletion | del A | 24487616 | 3175 | Unique | Tumor specific |
| Transition | a > G | 24487657 | 3216 | Unique | Tumor specific |
| Transition | g > A | 24487672 | 3231 | Unique | Hematopoietic linage |

NT - Unique National Centre for Biotechnology Information (NCBI) unique identifier code for contig,
Seq NT - position in SEQ ID NO 1,
GV - genetic variation,
A.A. - Amino acid.

TABLE 3
Recurrent and unique mutations in exons 3-4 and introns 3-4 of gHAS1 from
Waldenstom's Macroglobulinemia patients

| | | | | | | |
|---|---|---|---|---|---|---|
| Types of GV | GV | NT | Seq NT | Frequency | Comments | Effects |

Exon 3

| | | | | | | |
|---|---|---|---|---|---|---|
| Transition | g > A | 24488419 | 3978 | Unique | Tumor specific | Gly > Gly |
| Transition | t > C | 24488429 | 3988 | Unique | Germline origin | Cys > Arg |
| Transversions | t > A | 24488429 | 3988 | Unique | Tumor specific | Cys > Ser |
| Transition | t > C | 24488434 | 3993 | Unique | Germline origin | Val > Ala |
| Transition | t > C | 24488436 | 3995 | Unique | Germline origin | Cys > Cys |
| Transversions | a > T | 24488446 | 4005 | Recurrent | Tumor specific | Tyr > Phe |
| Transition | g > A | 24488455 | 4014 | Unique | Hematopoietic linage | Cys > Tyr |
| Transition | t > C | 24488456 | 4015 | Unique | Germline origin | Cys > Arg |
| Transition | t > C | 24488457 | 4016 | Unique | Hematopoietic linage | Ala > Ala |
| Transition | c > T | 24488458 | 4017 | Unique | Germline origin | Ala > Val |
| Transition | a > G | 24488470 | 4029 | Unique | Tumor specific | Asn > Ser |
| Transition | t > C | 24488503 | 4062 | Unique | Tumor specific | Phe > Ser |
| Transition | c > T | 24488522 | 4081 | Unique | Tumor specific | Leu > Leu |
| Transition | t > C | 24488523 | 4082 | Unique | Tumor specific | Pro > Pro |
| Transversions | t > G | 24488547 | 4106 | Unique | Tumor specific | Gly > Gly |
| Transversions | t > A | 24488560 | 4119 | Unique | Tumor specific | Val > Glu |
| Transversions | t > A | 24488581 | 4140 | Unique | Tumor specific | Val > Glu |
| Transition | g > A | 24488588 | 4147 | Unique | Germline origin | Val > Ile |
| Transition | g > A | 24488595 | 4154 | Unique | Germline origin | Leu > Leu |
| Transversions | g > C | 24488616 | 4175 | Unique | Tumor specific | Arg > Ser |
| Transition | a > G | 24488618 | 4177 | Unique | Tumor specific | Arg > Gly |
| Transition | t > C | 24488633 | 3978 | Unique | Hematopoietic linage | Cys > Arg |

Intron 3

| | | | | | |
|---|---|---|---|---|---|
| Transition | c > T | 24487855 | 3414 | Unique | Germline origin |
| Transition | a > G | 24487871 | 3430 | Unique | Germline origin |
| Transition | t > C | 24487878 | 3437 | Unique | Hematopoietic linage |
| Transition | t > C | 24487891 | 3450 | Unique | Tumor specific |
| Transition | g > A | 24487904 | 3463 | Recurrent | Germline origin |
| Transition | a > G | 24487940 | 3499 | Unique | Hematopoietic linage |
| Transition | t > C | 24487977 | 3536 | Unique | Tumor specific |
| Transversions | a > C | 24488015 | 3574 | Unique | Tumor specific |
| Transition | a > G | 24488046 | 3605 | Recurrent | Tumor specific |
| Transition | t > C | 24488112 | 3671 | Unique | Tumor specific |
| Transversions | t > A | 24488128 | 3687 | Recurrent | SNP-NCBI rs 11669079 |
| Transition | a > G | 24488137 | 3696 | Unique | Tumor specific |
| Transition | t > C | 24488140 | 3699 | Unique | Tumor specific |
| Transversions | g > T | 24488154 | 3713 | Unique | Tumor specific |
| Transversions | t > G | 24488194 | 3753 | Unique | Tumor specific |
| Transition | g > A | 24488209 | 3768 | Recurrent | SNP-NCBI rs 11084109 |
| Transition | t > C | 24488236 | 3795 | Unique | Tumor specific |
| Transition | g > A | 24488267 | 3826 | Recurrent | SNP-NCBI rs 11084110 |
| Transition | g > A | 24488344 | 3903 | Unique | Tumor specific |
| Transition | a > G | 24488355 | 3914 | Unique | Tumor specific |

Exon 4

| | | | | | | |
|---|---|---|---|---|---|---|
| Transversions | a > T | 24487724 | 3283 | Recurrent | novel SNP | Met > Leu |
| Transversions | g > C | 24487726 | 3285 | Recurrent | novel SNP | Arg > Pro |
| Transition | c > t | 24487764 | 3323 | Unique | Tumor specific | Thr > Thr |

Intron 4

| | | | | | |
|---|---|---|---|---|---|
| Transition | c > T | 24485577 | 1136 | Unique | Hematopoietic linage |
| Transition | c > T | 24485595 | 1154 | Unique | Germline origin |
| Transition | t > C | 24485630 | 1189 | Recurrent | Tumor specific |
| Transition | g > A | 24485631 | 1190 | Unique | Hematopoietic linage |
| Transition | c > T | 24485640 | 1199 | Unique | Tumor specific |
| Transversion | t > G | 24485795 | 1354 | Unique | Tumor specific |
| Insertion | x TTA | 24485814 | 1373 | Recurrent | Germline origin |
| Deletion | xTTTA | 24485817 | 1376 | Recurrent | Germline origin |
| Transition | t > C | 24485860 | 1419 | Unique | Tumor specific |
| Transition | g > A | 24485865 | 1424 | Unique | Germline origin |
| Transition | a > G | 24485873 | 1432 | Unique | Hematopoietic linage |
| Transition | t > C | 24485899 | 1458 | Unique | Tumor specific |
| Deletion | del C | 24485951 | 1510 | Unique | Tumor specific |
| Deletion | del T | 24485967 | 1526 | Recurrent | Tumor specific |
| Insertion | (T)s | 24485967 | 1526 | Recurrent | Germline origin |
| Transition | t > C | 24486013 | 1572 | Unique | Tumor specific |
| Deletion | CC | 24486041 | 1600 | Unique | Hematopoietic linage |
| Transition | t > C | 24486124 | 1683 | Unique | Tumor specific |
| Transversion | t > A | 24486135 | 1694 | Recurrent | SNP-NCBI rs 8103845 |
| Insertion | (T)s | 24486139 | 1698 | Recurrent | Germline origin |
| Transition | c > T | 24486141 | 1700 | Recurrent | SNP-NCBI rs 8104157 |
| Transition | a > G | 24486208 | 1767 | Unique | Tumor specific |
| Transition | c > T | 24486230 | 1789 | Unique | Tumor specific |
| Transition | a > G | 24486274 | 1833 | Unique | Tumor specific |
| Transition | a > G | 24486277 | 1836 | Unique | Tumor specific |
| Transition | t > C | 24486367 | 1926 | Unique | Tumor specific |
| Transversions | a > T | 24486408 | 1967 | Unique | Tumor specific |
| Transversions | g > t | 24486419 | 1978 | Recurrent | novel SNP |
| Transition | c > T | 24486461 | 2020 | Unique | Hematopoietic linage |
| Transition | a > G | 24486506 | 2065 | Unique | Germline origin |
| Transversions | c > G | 24486532 | 2091 | Recurrent | SNP-NCBI rs 4802848 |
| Transversions | c > A | 24486533 | 2092 | Recurrent | SNP-NCBI rs 4802849 |
| Deletion | delg | 24486723 | 2282 | Unique | Tumor specific |
| Transversions | g > T | 24486825 | 2384 | Unique | Germline origin |
| Transversions | g > C | 24486871 | 2430 | Recurrent | SNP-NCBI rs 4802850 |
| Transition | a > G | 24486916 | 2475 | Unique | Tumor specific |
| Transition | a > G | 24487039 | 2598 | Unique | Tumor specific |
| Transition | g > A | 24487089 | 2648 | Recurrent | SNP-NCBI rs 7254072 |
| Transversions | t > G | 24487116 | 2675 | Recurrent | SNP-NCBI rs 11667949 |
| Transversions | g > C | 24487148 | 2707 | Unique | SNP-NCBI rs 11667974 |
| Transition | a > G | 24487189 | 2748 | Unique | Germline origin |
| Transition | t > C | 24487193 | 2752 | Unique | Tumor specific |
| Transition | a > G | 24487213 | 2772 | Unique | Tumor specific |
| Transition | t > C | 24487234 | 2793 | Unique | Tumor specific |
| Transition | g > A | 24487242 | 2801 | Unique | Hematopoietic linage |
| Transition | a > G | 24487307 | 2866 | Unique | Tumor specific |
| Transition | a > G | 24487476 | 3035 | Unique | Tumor specific |
| Transition | a > G | 24487500 | 3059 | Unique | Tumor specific |
| Transition | c > T | 24487527 | 3086 | Unique | Tumor specific |
| Transition | a > G | 24487602 | 3161 | Unique | Hematopoietic linage |
| Transition | t > C | 24487623 | 3182 | Unique | Tumor specific |
| Transition | g > a | 24487661 | 3220 | Unique | Tumor specific |

Seq NT - position relative to SEQ ID NO 1,
GV - genetic variation,
A.A. - Amino acid.

Diagnostic Application

T The identification of recurrent patterns of genetic variations in genomic HAS1 that characterize cancer cells and are absent from healthy cells (as reported in the NCBI database) provides a cancer cell marker that can be used to detect predisposition to malignancy or malignant cells. In this context, the term “recurrent” is defined as a newly identified genetic variation(s) that is found in more than one patient. Since genomic DNA is very stable, a diagnostic test detecting genetic variations is feasible on samples that must be stored for hours or days or those that are shipped from distant locations for testing. Genetic variations can be tested as single representative variations that define the entire recurrent HAS1 “haplotype” in a population or in individual cells. Alternatively, a battery of simultaneous or sequential tests for multiple variations, particularly well suited for use in association with a microfluidic device, can be used to determine whether or not the recurrent pattern (henceforth referred to as the HAS1 “haplotype”) is present in a population of cells or

The existence of specific, recurrent HAS1 haplotypes in MM and WM, and likely in other cancers or proliferative diseases or disorders, and in particular those characterized by abnormal HASs; provide markers to identify malignant cells and to distinguish between malignant and non-malignant cells. A predisposition to cancer or proliferative diseases or disorders may be ascertained by testing mammalian biological samples for the presence of the HAS1 genomic mutations disclosed herein in general and in Table 2, Table 3, and in particular Tables 4, 5, 6 and 7. This predisposition can be determined by testing DNA from cells removed from any tissue or fluid from the mammal in general or in particular from tissues not involved in the disease (for example buccal cells), from cells of the haematopoietic lineage (for example T cells or polymorphonuclear cells in MM and WM) or from cells thought to be malignant (for example B-Cells in MM and WM), to detect the presence of the genomic variations described above. Combinations of tests detecting the described categories of genetic variation (germline origin, hematopoietic lineage or tumor specific) provide a staging strategy to identify germline predisposition, high risk hematopoietic involvement and frank malignancy, as well as for monitoring response to therapy of malignant cells.

After sequence analysis of exons 3-4 and introns 3 and 4 of the HAS1 gene from 5 WM patients, a number of GV have been detected including tranversions and transitions, deletions and insertions. Among these GV recurrent and unique (specific to individual patients) mutations have been identified, the latter of which are transitions. The reason transitions are more common is indicative of the underlying causes of mutations and to the size of the bases. A purine can be altered so that it base pairs like the other. It is impossible for a purine to be altered to resemble a pyrimidine, or vice versa.

The present invention encompasses any method to detect individual or multiple of the described genetic variation(s) in individual cells or in populations of cells, including but not restricted to allelic discrimination methods, SNP detection methods, PCR and single cell PCR. It also encompasses in situ PCR for detection of DNA encoding the HAS1 protein. The technique is preferred when the copy number of a target nucleic acid is very low, or when different forms of nucleic acids must be distinguished. The method is especially important in detecting and differentiating pre-cancer and cancer cells from normal cells. The method is also useful in detecting subsets of cells destined to become cancer cells. Confirmation of in situ PCR product identity is accomplished by in situ hybridization with a nested 32P-labeled probe or by examining the products using Southern blot analysis to corroborate predicted base pair size.

Mutational Patterns

1. Disease specific: The tumor specific genetic variations (mutations, substitutions, deletions, insertions) are the somatic genetic variations that are detected in B cell linage cells (the malignant cells) of the patients (Table 4). These mutations are associated with MM and/or WM.

TABLE 4
Disease Specific Genetic Markers

| | | | | | |
|---|---|---|---|---|---|
| Types of GV | GV | NT | Seq NT | Frequency | Effects |

Exon 3

| | | | | | | |
|---|---|---|---|---|---|---|
| Transition | g > A | 24488419 | 3978 | Unique | Gly > Gly | WM |
| Transversion | t > A | 24488429 | 3988 | Recurrent | Cys > Ser | MM/ |
| | | | | | | WM |
| Transition | t > C | 24488436 | 3995 | Unique | Cys > Cys | MM |
| Trans- | a > T | 24488446 | 4005 | Recurrent | Tyr > Phe | WM |
| versions | | | | | | |
| Transition | a > G | 24488470 | 4029 | Unique | Asn > Ser | WM |
| Transition | t > C | 24488503 | 4062 | Unique | Phe > Ser | WM |
| Transition | c > T | 24488505 | 4064 | Unique | Ser > Ser | MM |
| Transition | c > T | 24488517 | 4076 | Unique | Asp > Asp | MM |
| Transition | c > T | 24488522 | 4081 | Unique | Leu > Leu | WM |
| Transition | t > C | 24488523 | 4082 | Unique | Pro > Pro | WM |
| Trans- | t > G | 24488547 | 4106 | Unique | Gly > Gly | WM |
| versions | | | | | | |
| Transition | t > C | 24488553 | 4112 | Unique | Ala > Ala | MM |
| Trans- | t > A | 24488560 | 4119 | Unique | Val > Glu | WM |
| versions | | | | | | |
| Transition | c > T | 24488564 | 4123 | Unique | Arg > Trp | MM |
| Trans- | t > A | 24488581 | 4140 | Unique | Val > Glu | WM |
| versions | | | | | | |
| Transition | c > T | 24488608 | 4167 | Unique | Pro > Leu | MM |
| Transv- | g > C | 24488616 | 4175 | Unique | Arg > Ser | WM |
| ersions | | | | | | |
| Transition | a > G | 24488618 | 4177 | Unique | Arg > Gly | WM |

Intron 3

| | | | | | |
|---|---|---|---|---|---|
| Transition | t > C | 24487891 | 3450 | Unique | WM |
| Transition | t > C | 24487977 | 3536 | Unique | WM |
| Trans- | a > C | 24488015 | 3574 | Unique | WM |
| versions | | | | | |
| Transition | a > G | 24488046 | 3605 | Recurrent | WM |
| Transition | t > C | 24488112 | 3671 | Unique | WM |
| Transition | a > G | 24488137 | 3696 | Unique | WM |
| Transition | t > C | 24488140 | 3699 | Unique | WM |
| Transition | t > C | 24488141 | 3700 | unique | MM |
| Trans- | g > T | 24488154 | 3713 | Unique | WM |
| versions | | | | | |
| Trans- | t > G | 24488194 | 3753 | Unique | WM |
| versions | | | | | |
| Transition | t > C | 24488236 | 3795 | Unique | WM |
| Transition | a > G | 24488320 | 3879 | Unique | MM |
| Transition | g > A | 24488344 | 3903 | Unique | WM |
| Transition | a > G | 24488355 | 3914 | Unique | WM |
| Transversion | g > T | 24488395 | 3954 | Unique | MM |
| Transversion | a > T | 24488408 | 3967 | Unique | MM |

Exon 4

| | | | | | | |
|---|---|---|---|---|---|---|
| Transition | c > t | 24487764 | 3323 | Unique | Thr > Thr | WM |

Intron 4

| | | | | | |
|---|---|---|---|---|---|
| Transition | a > G | 24485564 | 1123 | Unique | MM |
| Transition | t > C | 24485574 | 1133 | Unique | MM |
| Transition | a > G | 24485594 | 1153 | Unique | MM |
| Transition | t > C | 24485630 | 1189 | Recurrent | WM |
| Transition | c > T | 24485640 | 1199 | Unique | WM |
| Transition | a > G | 24485658 | 1217 | Unique | MM |
| Transition | t > C | 24485663 | 1222 | Unique | MM |
| Deletion | del C | 24485686 | 1245 | Unique | MM |
| Transition | a > G | 24485727 | 1286 | Unique | MM |
| Transversion | t > G | 24485795 | 1354 | Unique | WM |
| Transition | t > C | 24485797 | 1356 | Unique | MM |
| Transition | g > A | 24485802 | 1361 | Unique | MM |
| Transition | g > A | 24485805 | 1364 | Unique | MM |
| Transition | g > A | 24485844 | 1403 | Unique | MM |
| Transition | t > C | 24485860 | 1419 | Unique | WM |
| Transition | t > C | 24485899 | 1458 | Recurrent | MM/ |
| | | | | | WM |
| Transversion | t > G | 24485936 | 1495 | Unique | MM |
| Deletion | del C | 24485951 | 1510 | Recurrent | MM/ |
| | | | | | WM |
| Deletion | del T | 24485967 | 1526 | Recurrent | WM |
| Transition | a > G | 24486002 | 1561 | Unique | MM |
| Transition | t > C | 24486013 | 1572 | Unique | WM |
| Transition | a > G | 24486015 | 1574 | Unique | MM |
| Transition | t > C | 24486124 | 1683 | Unique | WM |
| Transition | a > G | 24486208 | 1767 | Unique | WM |
| Transition | c > T | 24486230 | 1789 | Unique | WM |
| Transition | t > C | 24486244 | 1803 | Unique | MM |
| Transition | a > G | 24486274 | 1833 | Unique | WM |
| Transition | a > G | 24486277 | 1836 | Unique | WM |
| Transition | t > C | 24486367 | 1926 | Unique | WM |
| Trans- | a > T | 24486408 | 1967 | Unique | WM |
| versions | | | | | |
| Deletion | delg | 24486723 | 2282 | Unique | WM |
| Transition | t > C | 24486747 | 2306 | Unique | MM |
| Transversion | g > T | 24486773 | 2332 | Unique | MM |
| Transition | a > G | 24486815 | 2374 | Unique | MM |
| Transition | a > G | 24486916 | 2475 | Unique | WM |
| Transition | a > G | 24486943 | 2502 | Unique | MM |
| Transition | a > G | 24487004 | 2563 | Unique | MM |
| Transition | a > G | 24487039 | 2598 | Unique | WM |
| Transition | t > C | 24487126 | 2685 | Unique | MM |
| Transition | t > C | 24487140 | 2699 | Unique | MM |
| Transition | t > C | 24487156 | 2715 | Unique | MM |
| Transversion | a > C | 24487185 | 2744 | Unique | MM |
| Transition | c > T | 24487188 | 2747 | Unique | MM |
| Transition | t > C | 24487193 | 2752 | Unique | WM |
| Transition | a > G | 24487213 | 2772 | Unique | WM |
| Transition | a > G | 24487214 | 2773 | Unique | MM |
| Transition | t > C | 24487234 | 2793 | Unique | WM |
| Transition | c > T | 24487283 | 2842 | Unique | MM |
| Transition | a > G | 24487307 | 2866 | Unique | WM |
| Transition | a > G | 24487476 | 3035 | Unique | WM |
| Transition | a > G | 24487493 | 3052 | unique | MM |
| Transition | a > G | 24487500 | 3059 | Unique | WM |
| Transition | c > T | 24487527 | 3086 | Unique | WM |
| Deletion | del A | 24487616 | 3175 | Unique | MM |
| Transition | t > C | 24487623 | 3182 | Unique | WM |
| Transition | a > G | 24487657 | 3216 | Unique | MM |
| Transition | g > a | 24487661 | 3220 | Unique | WM |
| Deletion | del T | 24485969 | 1528 | recurrent | MM |

These somatic, tumor specific genetic variations provide markers for use in diagnosis and/or monitoring of the disease. They can be used to detect malignant cells at the time of diagnosis and/or during progression of the disease, as a marker for existing disease or as an early marker for emerging disease.

2. Hematopoietic involvement: Genetic variations that are specific to the hematopoietic linage of the patients. These mutations are detected in hematopoietic progenitor cells (stem cells), T cells and other hematopoietic cell types that comprise the healthy hematopoietic cells of the patient (Table 5). They are not germline mutations, as defined by their absence from a representative tissue having the germline sequence, in this case buccal cells (epithelial cells of the patients that are nonmalignant). The mutations identified as being specific to the hematopoietic lineage are detected in hematopoietic cells but not germline tissues (in this case in buccal cells) from patients we have analyzed to date. They are absent from healthy donor hematopoietic cells whose HAS1 gene segments have been sequenced by the inventors and they have not been reported in the NCBI database.

TABLE 5
Early Stage Markers of Disease

| | | | | | |
|---|---|---|---|---|---|
| Types of | | | | | |
| GV | GV | NT | Seq NT | Frequency | Effects |

Exon 3

| | | | | | | |
|---|---|---|---|---|---|---|
| Transition | t > C | 24488434 | 3993 | Recurrent | Val > Ala | MM |
| Transition | g > A | 24488455 | 4014 | Recurrent | Cys > Tyr | MM/ |
| | | | | | | WM |
| Transition | t > C | 24488457 | 4016 | Recurrent | Ala > Ala | MM/ |
| | | | | | | WM |
| Transition | t > C | 24488467 | 4026 | Unique | Val > Ala | MM |
| Transition | a > G | 24488575 | 4134 | Unique | Asp > Gly | MM |
| Transition | t > C | 24488633 | 4192 | Unique | Cys > Arg | WM |

Intron 3

| | | | | | |
|---|---|---|---|---|---|
| Transition | t > C | 24487878 | 3437 | Unique | WM |
| Transition | a > G | 24487940 | 3499 | Unique | WM |
| Transition | g > A | 24488191 | 3750 | unique | MM |
| Transition | g > A | 24488249 | 3808 | unique | MM |
| Transition | g > A | 24488303 | 3862 | unique | MM |
| Transition | a > G | 24488361 | 3920 | recurrent | MM |
| Transition | g > a | 24488374 | 3933 | Unique | MM |

Exon 4

| | | | | | | |
|---|---|---|---|---|---|---|
| Transversion | a > T | 24487724 | 3283 | Recurrent | Met > Leu | MM/ |
| | | | | | | WM |
| Transversion | g > C | 24487726 | 3285 | Recurrent | Arg > Pro | MM/ |
| | | | | | | WM |

Intron 4

| | | | | | |
|---|---|---|---|---|---|
| Transition | c > T | 24485577 | 1136 | Unique | WM |
| Transition | g > A | 24485631 | 1190 | Unique | WM |
| Transition | t > C | 24485767 | 1326 | Unique | MM |
| Transversion | g > T | 24485780 | 1339 | unique | MM |
| Transition | t > C | 24485801 | 1360 | recurrent | MM |
| Transition | a > G | 24485873 | 1432 | Unique | WM |
| Transition | t > C | 24486030 | 1589 | Unique | MM |
| Deletion | CC | 24486041 | 1600 | Unique | WM |
| Transition | t > c | 24486116 | 1675 | Unique | MM |
| Transversion | g > T | 24486419 | 1978 | Recurrent | MM/ |
| | | | | | WM |
| Transition | c > T | 24486461 | 2020 | Unique | WM |
| Transition | a > G | 24486494 | 2053 | Unique | MM |
| Transversion | a > C | 24486576 | 2135 | Unique | MM |
| Transition | t > C | 24486744 | 2303 | unique | MM |
| Transition | a > G | 24486788 | 2347 | Unique | MM |
| Transition | t > C | 24486829 | 2388 | Unique | MM |
| Transition | g > A | 24487120 | 2679 | Unique | MM |
| Transition | g > A | 24487242 | 2801 | Unique | WM |
| Transition | g > A | 24487278 | 2837 | unique | MM |
| Transition | g > A | 24487556 | 3115 | Unique | MM |
| Transition | t > C | 24487579 | 3138 | unique | MM |
| Transition | a > G | 24487602 | 3161 | Unique | WM |
| Transition | g > A | 24487672 | 3231 | Unique | MM |

Mutations specific for cells within the hematopoietic lineage of the patients are useful markers for advanced predisposition to malignant disease or impending disease, and can thus be used for diagnosis and monitoring of patients during, for example, “watchful waiting” or as part of continuing monitoring of individuals thought to be at risk of cancer. Individuals with the genetic variations specific to the hematopoietic lineage may be at greater risk and thus require more frequent monitoring than those individuals having only germline genetic variations (see below). They are markers of a second stage of genetic variation that has advanced beyond the germline set of predisposing genetic variations. Most likely, accumulation of these mutations accompany development of MM and/or WM, as evidenced by their presence in the HAS1 gene segments in healthy tissues from these patients.

3. Germline mutations: These mutations are detected in all cells of an individual, in this case a patient, using buccal cells (of the epithelial lineage) as a representative healthy tissue (Table 6). These mutations can also be found in B, T, plasma cells (PC) and stem cells from patients because they are representative of the patient germline that is present in all cells of the body. However, these mutations are absent from cells of healthy donors whose HAS1 gene has been sequenced as disclosed herein, and though reported in the NCBI database have not been previously associated with predisposition to disease. These germline genetic variations predispose individuals to cancer as indicated by their presence in MM and/or WM patients but not in healthy donors (this means these genetic variations are more frequent in patient populations as compared to healthy individuals).

TABLE 6
Predisposition to Disease Specific Genetic Markers

| Types | | | | | | |
|---|---|---|---|---|---|---|
| of GV | GV | NT | Seq NT | Frequency | Effects | |
| Transition | g > A | 24488419 | 3978 | Unique | Gly > Gly | MM |
| Transition | c > T | 24488458 | 4017 | Unique | Ala > Val | MM |
| Transition | t > C | 24488429 | 3988 | Unique | Cys > Arg | WM |
| Transition | t > C | 24488434 | 3993 | Unique | Val > Ala | WM |
| Transition | t > C | 24488436 | 3995 | Unique | Cys > Cys | WM |
| Transition | t > C | 24488456 | 4015 | Unique | Cys > Arg | WM |
| Transition | c > T | 24488458 | 4017 | Unique | Ala > Val | WM |
| Transition | g > A | 24488588 | 4147 | Unique | Val > Ile | WM |
| Transition | g > A | 24488595 | 4154 | Unique | Leu > Leu | WM |

Intron 3

| | | | | | |
|---|---|---|---|---|---|
| Transition | a > G | 24488147 | 3706 | unique | MM |
| Transition | c > T | 24488405 | 3964 | Unique | MM |
| Transition | c > T | 24487855 | 3414 | Unique | WM |
| Transition | a > G | 24487871 | 3430 | Unique | WM |
| Transition | g > A | 24487904 | 3463 | Recurrent | WM |

Intron 4

| | | | | | |
|---|---|---|---|---|---|
| Transition | c > T | 24485595 | 1154 | Unique | WM |
| Transition | g > A | 24485621 | 1180 | unique | MM |
| Insertion | xTTTA | 24485814 | 1373 | Unique | MM/ |
| | | | | | WM |
| Deletion | x TTA | 24485817 | 1376 | recurrent | MM/ |
| | | | | | WM |
| Transition | g > A | 24485865 | 1424 | Unique | WM |
| Insertion | (T)s | 24485967 | 1526 | recurrent | MM/ |
| | | | | | WM |
| Insertion | (T)s | 24486139 | 1698 | Recurrent | WM |
| Insertion | (Ts) | 24486140 | 1699 | recurrent | MM |
| Insertion | (Ts) | 24486142 | 1701 | recurrent | MM |
| Transition | a > G | 24486506 | 2065 | Unique | WM |
| Transition | a > G | 24486671 | 2230 | unique | MM |
| Trans- | g > T | 24486825 | 2384 | Unique | WM |
| versions | | | | | |
| Transition | g > A | 24487010 | 2569 | unique | MM |
| Transition | a > G | 24487189 | 2748 | Unique | WM |
| Transition | t > C | 24487227 | 2786 | unique | MM |
| Transition | t > C | 24487459 | 3018 | unique | MM |
| Transition | a > G | 24487577 | 3136 | unique | MM |
| Transition | t > C | 24487588 | 3147 | Unique | MM |

Identifying germline genetic variations in an individual who is not yet a “patient” provides a test for predisposition to MM and/or WM, and a means to identify and monitor individuals at risk of developing disease. Such monitoring provides a test to identify “at risk” individuals. Such identification will facilitate the development of preventive strategies and their application only to those individuals at risk. Knowledge of predisposing mutations may enable prevention in the general population or new therapeutic strategies, by identifying those individuals most likely to benefit. Cost considerations and potential side effects would prevent the use of preventive strategies in all individuals, making an identification strategy a critical and essential element of future disease prevention therapies.

Therefore, these germline genetic variations are predisposing elements for MM and/or WM and can be used for predictive or preventive monitoring strategies.

Together, testing for tumor specific, hematopoietic, and germline genetic variations provides a testing sequence for increasing predisposition to disease and for use as an early maker of emerging malignancy. After identification of “at risk” individuals with germline genetic variations that predispose to cancer, these identified individuals can be followed at regular time points for early detection of emerging hematopoietic lineage mutations, and at a later stage to detect emergence of tumor specific mutations. These events are likely to occur prior to pathological detection of frank malignancy and thus provide a set of valuable early markers for regular follow up of potential patients at risk of developing cancer.

4. Single Nucleotide Polymorphisms (SNPs): SNPs are germline genetic variations detected in every single cell in the body of a given individual, including buccal cells, B, T, PC, stem cells. A genetic variation (mutation) is defined as a SNP if it has a defined frequency in a population of individuals. By definition a polymorphism must be present in more than one individual. Some SNPs in the HAS1 gene are also reported in the NCBI database and are not novel. However, our data showing that these SNPs can be used as markers for identifying individuals at risk of MM and/or WM is novel, as is the observation of the inventors that these SNPs are present at a greatly increased frequency in patient with MM and WM. In these patients we detected increased homozygosity for the HAS1 SNPs reported in NCBI was defined, therefore this work is the first to show that most WM and MM patients are homozygous for these genetic variations.

For the instances reported here, they are frequent in patient populations suffering from cancer or prolfierative disease or disorder but not in healthy individuals.

TABLE 7
Novel SNPs

| | | | | | |
|---|---|---|---|---|---|
| Types of GV | GV | NT | Seq NT | Frequency | Effects |

Exon 3

| | | | | | | |
|---|---|---|---|---|---|---|
| Transition | t > C | 24488429 | 3988 | Recurrent | Cys > Arg | MM/ |
| | | | | | | WM |

Exon 4

| | | | | | | |
|---|---|---|---|---|---|---|
| Trans- | a > T | 24487724 | 3283 | Recurrent | Met > Leu | WM |
| versions | | | | | | |
| Trans- | g > C | 24487726 | 3285 | Recurrent | Arg > Pro | WM |
| versions | | | | | | |

Intron 4

| | | | | | |
|---|---|---|---|---|---|
| Trans- | g > t | 24486419 | 1978 | Recurrent | WM |
| versions | | | | | |
| Transition | c > T | 24485595 | 1154 | recurrent | MM |
| Transition | c > T | 24485793 | 1352 | recurrent | MM |
| Transition | a > G | 24486597 | 2156 | recurrent | MM |

Unique genetic variations may also become classified as SNPs if additional screening of more individuals indicate these have a definable frequency within the general population.

In the method for diagnosing the existence of cancer or a prolfierative disease or disorder, or a predisposition to cancer or a prolfierative disease or disorder; a genomic nucleic acid sequence isolated from a biological sample taken from a mammal is contacted with the nucleic acid sequence or portion thereof encoding an intronic or exonic genetic variation which is an early marker for cancer or a prolfierative disease or disorder, under stringent conditions that allow hybridization between the sequences and detecting the hybridized sequences. The presence of a genomic nucleic acid sequence or the presence of an altered genomic nucleic acid sequence as compared to a normal nucleic acid sequence is indicative of cancer or a prolfierative disease or disorder, or a predisposition thereto, in the mammal. The increased presence of the DNA, mRNA and/or alternate splice forms of the mRNA in the biological sample is indicative of cancer or a prolfierative disease or disorder, or a predisposition thereto.

### Example 1

### Diagnosis of Patients Through B-Cell HAS1 Haplotype Determination

A blood sample is provided by a human or other mammalian patient from which B-cells are purified and separated by means known to those skilled in the art. One non-limiting example of such separation and purification means is Fluorescence Activated Cell Sorting (FACS) purification and separation using B-Cell specific antibody (for example including, but not limited to mouse-antihuman CD20) with a flurophore conjugated antibody specific to the B-cell specific antibody (for example including, but not limited to, Flourescein:goat-antimouse antibody). B-cells are then subjected to lysis sufficient to release genomic DNA such means well known in the art and including, but not limited to ultrasonic lysis, heat lysis or Sodium Docenyl Supphate (SDS) lysis. See for example Sambrook et al. Primer: A Laboratory Manual Carl Dieffenbach Ed. Cold Spring Harbor Press (1995).

The presence and quantity of genomic DNA carrying WM or MM in specific, or cancer in general mutations (as disclosed herein in general and in Tables 2 and 3, and in particular Tables 4, 5, 6 and 7) is determined using means known in the art including but not limited to Quantitative PCR, PCR-based DNA sequencing or PCR in general, restriction endonuclease fragment hybridization using mutation specific probes (following or independent of PCR amplification or Restriction Fragment Length Polymorphism) hybridization under stringent conditions with allele specific oligonucleotides (ASO hybridization) of tagged probes, SNP microarray assay or hybridization of labeled DNA or RNA probes (including chemical variants thereof capable of hybridization to genomic DNA and such hybridization being detectable). See for example Sambrook et al. Molecular Cloning a Laboratory Manual Cold Spring Harbor Press 2ed. (1989) or PCR Primer: A Laboratory Manual Carl Dieffenbach Ed. Cold Spring Harbor Press (1995).

The quantity of MM, WM or cancer related genetic mutations (as disclosed herein) compared to total B-cell genomic content is determined and used to assess the prevalence of genetically predisposed B-cells, state of disease progression, metastasis progression, relapse of disease, remission of disease, response of the patient to treatment/chemotherapy, and other beneficial determinations known to those skilled in the art. One can test for a single GV as disclosed herein or a combination of at least two GV as disclosed herein, amounting to the ability to use any of the multiple potential sets of GVs as a cancer or proliferative disease or disorder monitoring tools.

### Example 2

### Single Cell Analysis and Frequency of Mutation of Analysis

A blood sample is provided by a human or other mammalian patient from which B-cells are purified and separated by means known to those skilled in the art. One non-limiting example of such separation and purification means is Fluorescence Activated Cell Sorting (FACS) purification and separation using B-Cell specific antibody (for example including, but not limited to mouse-antihuman CD20) with a fluorophore conjugated antibody specific to the B-cell specific antibody (for example including, but not limited to, Flourescein:goat-antimouse antibody). Individual B-cells are then subjected to lysis sufficient to release genomic DNA such means well known in the art and including, but not limited to ultrasonic lysis, heat lysis or Sodium Docenyl Supphate (SDS) lysis. See for example Sambrook et al. Molecular Cloning a Laboratory Manual Cold Spring Harbor Press 2ed. (1989) or PCRPrimer: A Laboratory Manual Carl Dieffenbach Ed. Cold Spring Harbor Press (1995).

The presence of genomic DNA carrying WM or MM in specific, or cancer in general, mutations (as disclosed herein in general and in Table 2, Table 3, in particular Tables 4, 5, 6 and 7) is determined using means known in the art including but not limited to Single Cell PCR, generally being PCR with particularly high fidelity in replication and sequencing restriction endonuclease fragment hybridization using mutation specific probes (following or independent of PCR amplification or Restriction Fragment Length Polymorphism), hybridization with allele specific oligonucleotides (ASO hybridization) of tagged probes, SNP microarray assay or hybridization of labeled DNA or RNA probes (including chemical variants thereof capable of hybridization to genomic DNA and such hybridization being detectable). See for example Sambrook et al. Molecular Cloning a Laboratory Manual Cold Spring Harbor Press 2ed. (1989) or PCRPrimer: A Laboratory Manual Carl Dieffenbach Ed. Cold Spring Harbor Press (1995).

Such methods are particularly well suited to the use of microfluidic Devices (as defined herein and generally known in the art). The presence or absence of MM, WM or cancer related genetic mutations (as disclosed herein) is determined and the frequency of the presence of the mutations used to assess the prevalence of genetically predisposed B-cells, state of disease progression, metastasis progression, relapse of disease, remission of disease, response of the patient to treatment/chemotherapy, and other beneficial determinations known to those skilled in the art. In particular this information could be useful for observation and determination of human or mammalian patients progressing from a normal to malignant state of disease, for example detecting progression to WM or MM by observing the presence of WM or MM specific genetic mutations (as disclosed herein in general and Tables 2 and 3, and in particular Tables 4, 5, 6 and 7). Alternatively, the transition from a remissive state of MM to a progressive or relapsed state of MM can be determined using blood samples from the human or mammalian patient and detection of specific genetic mutations (as disclosed herein in general and Tables 2 and 3, and in particular Tables 4, 5, 6 and 7). Example 3: Genetic Tagging Strategy

The method of genetic tagging can be used for identification and induction (if necessary) of point mutations in the genomic sequence and is disclosed more particularly in United States Patent Application #20030119190.; which describes the use of a non-replicating retroviral vector is used as a transporter for randomly introducing mutations into the genome of a host cell. The viral sequence also functions as a tag to identify the mutated gene. Genetic tagging strategy offers the following advantages over other mutagenesis techniques:

1) It can identify and induce point mutations in a localized and controlled manner.

2) It can be used in both uni-celluar and multi-cellular organisms.

3) One can amplify mutated genes from a heterogeneous DNA sample by PCR-based techniques.

4) It is possible to identify, and clone novel genes.

Detection methods for SNP genotyping which can be adapted for point mutation detection and include, but are not limited to, indirect colorimetric, mass spectrometry, fluorescence, fluorescence resonance energy transfer, fluorescence polarization, chemiluminescence. These methods involve hybridization with allele specific probes, oligonucleotide ligation, single nucleotide primer extension, enzymatic cleavage. One skilled in the art would be able to assess the benefits and disadvantages of each method for the particular sample being tested depending on sample quality, quantity and needed accuracy.

### Example 4

### Therapeutics

The present invention discloses novel genetic mutation indicative of a predisposition to disease and particular disease state (malignancy), in particular MM and WM and more generally cancer. These genetic mutation are further proposed herein to alter RNA splicing. Therefore, use of compounds or factors capable of interfering, inhibiting or otherwise reducing the aberrant RNA splicing resulting in WM, MM, cancer, and proliferative diseases or disorders; specific HAS isoforms disclosed herein and otherwise known in the art, represents a therapeutic target for cancer therapies in general and WM and MM therapies in specific.

Such compounds, factors or methodologies are known to those skilled in the art and include, but are not limited to:
1) Targeting splicing factors. Use of agents that target splicing factors or enzymes that modify splicing factors. See for example United States Patent Application No. 20050053985, Sun, H. et al. Mol Cell Bio 20:6414 (2000), and Villemaire, J. et al J Biol Chem 278:5031 (2003).
2) Gene-specific therapy can be mediated by oligonucleotides or oligonucleotide-like compounds. Gene-specificity can be accomplished by targeting the oligonucleotides by base pairing to the desired transcript and to specific cis-acting elements within the transcript. Oligonucleotide-based therapies can be used to inhibit or to activate specific splicing events either by binding an element and sterically blocking its activity or by binding an element and recruiting other effector molecules to this site. See for example United States Patent Application Nos. 20020068321, and 20020038007.
3) Bifunctional oligonucleotides. This method has been named TOES and/or TOSS (targeted oligonucleotide enhancer/silencer of splicing). Bifunctional reagents contain an antisense targeting domain and an effector domain, which either silences or activates a targeted exon or intron. The effector domain of these oligomers will be peptides with 5, 10 or 15 arginine-serine (RS i.e. splicing factor) repeats, the activity of which was predicated. The effector fluction of these oligomers will be mediated by indirect recruitment of splicing factors via their binding sites. See for example Matter, N. et al. Nuci Acid Res 33:e41 (2005),
4) Isoform-specific RNAI. The use of exon (intron)-specific RNA interference (RNAi). This system can effectively and specifically knock down transcript levels. See for example Zhang, L. et al. Cancer Biol Ther 15:3 (2004).
5) RNA-based corrective therapy and genetic repair strategies. A group of methodologies that have been developed to reprogram mRNAs can be used to modify the outcome of alternative splicing decisions. RNA reprogramming can be achieved at multiple sites during the process of gene expression. The earliest target for RNA revision is the nascent primary transcript, where alternative splicing reactions can be redirected to preferentially express certain isoforms over others by changing secondary structure of pre-mRNA which can be achieved through mutating and disrupting stem -loop structures. See for example Kapsa, R. M. et al. Gene Ther 9:695 (2002).

In contrast to the traditional approach to gene therapy, genetic repair strategies attempt to directly correct endogenous genetic mistakes rather than deliver extra copies of genes to cells. Genetic repair strategies attempt to repair defective instructions in a site-specific manner. Processes such as homologous recombination and DNA mismatch repair methods can be used to repair mutant DNA in a site-specific manner.

In the method of treatment, the administration of the oligonucleotides of the invention may be provided prophylactically or therapeutically. The oligonucleotide or mixtures thereof may be provided in a unit dose form, each dose containing a predetermined quantity of oligonucleotides calculated to produce the desired effect in association with a pharmaceutically acceptable diluent or carrier such as phosphate-buffered saline to form a pharmaceutically composition. In addition, the oligonucleotide may be formulated in solid form and redissolved or suspended prior to use. The pharmaceutical composition may optionally contain other chemotherapeutic agents, antibodies, antivirals, exogenous immunomodulators or the like.

The route of administration may be intravenous, intramuscular, subcutaneous, intradermal, intraperitoneal, intrathecal, ex vivo, and the like. Administration may also be by transmucosal or transdermal means, or the compound may be administered orally. For transmucosal or transdermal administration, penetrants appropriate to the barrier to be permeated as used in the formulation. Such penetrants are generally known in the art, and include, for example, for transmucosal administration bile salts and fusidic acid derivatives. In addition, detergents may be used to facilitate permeation. Transmucosal administration may be through nasal sprays, for example, or using suppositories. For oral administration, the oligonucleotides are formulated into conventional oral administration forms, such as capsules, tablets and tonics. For topical administration, the oligonucleotides of the invention are formulated into ointments, salves, gels, or creams, as is generally known in the art.

In providing a mammal with the compounds or factors of the present invention, preferably a human, the dosage of administered compounds or factors will vary depending upon such factors as the mammal's age, weight, height, sex, general medical condition, previous medical history, disease progression, tumor burden, and the like. Other therapeutic drugs may be administered in conjunction with the compounds or factors.

The efficacy of treatment using the compounds or factors may be assessed by determination of alterations in the presence and quantity of HAS1 genomic DNA containing the mutations as disclosed herein, the concentration or activity of the DNA gene product of the HAS1 isoforms, tumor regression, or a reduction of the pathology or symptoms associated with the cancer.

### Example 5

### Individual Patient Based Disease Monitoring

As disclosed herein in general, and Table 2 and Table 3 in specific, there exist a number of patient specific genomic DNA mutations observed in the HAS1 gene; for cancer patients in general and for MM and WM in particular. Therefore, one skilled in the art is enabled by the present invention to obtain patient specific disease markers allow the monitoring of therapy efficacy, disease state, malignancy presence or state of remission in the patient. One potential methodology would be available to one skilled in the art is as immediately follows:

Prior to, during or following treatment, a human or mammalian patient provides a blood sample from which cells are purified, for example B-cells, as described in Example 1 and Example 2 above. Genomic DNA is isolated and the HAS1 gene sequenced. Sequencing of genomic DNA is well known in the art, both from cell populations or from individual cells: see for example Sambrook et al. Molecular Cloning a Laboratory Manual Cold Spring Harbor Press 2ed. (1989) or PCR Primer: A Laboratory Manual Carl Dieffenbach Ed. Cold Spring Harbor Press (1995).

From the sequence information, both individual specific and disease specific mutations, as taught herein in general and in Tables 2 and 3 in particular, are catalogued. Using the methods described in Example 1 and Example 2 above, the continued presence and/or frequency of occurrence of the genomic mutations may be observed during the course of treatment; specifically prior to, during or after administration of a therapeutic or therapeutic regimen.

While particular embodiments of the present invention have been described in the foregoing, it is to be understood that other embodiments are possible within the scope of the invention and are intended to be included herein. It will be clear to any person skilled in the art that modifications of and adjustments to this invention, not shown, are possible without departing from the spirit of the invention as demonstrated through the exemplary embodiments. The invention is therefore to be considered limited solely by the scope of the appended claims.

What is claimed is:

1. A method to diagnose the presence of a proliferative disease or disorder in a patient comprising determining the presence of a genetic mutation in a population of cells obtained from the patient, the genetic mutation selected from Table 4 and Table 5.

2. The method of claim 1 wherein the proliferative disease or disorder is cancer.

3. The method of claim 1 wherein the proliferative disease or disorder is WM.

4. The method of claim 2 wherein the cancer is MM.

5. The method of claim 1 wherein the population of cells is a subpopulation of cells with desired characteristics isolated form the patient.

6. The method of claim wherein the subpopulation of cells comprises B-cells.

7. The method of claim 3 wherein the subpopulation of cells comprises T-cells.

8. A method to diagnose the predisposition to a proliferative disease or disorder in a patient comprising determining the presence of a genetic mutation in a population of cells obtained from the patient, the genetic mutation selected from Table 6 and Table 7.

9. The method of claim 8 wherein the proliferative disease or disorder is cancer.

10. The method of claim 8 wherein the proliferative disease or disorder is MM.

11. The method of claims 8 wherein the proliferative disease or disorder is WM.

12. A method of monitoring the progress of a proliferative disease or disorder in a patient comprising the steps of:
A) Obtaining a population of cells from a patient at at least two points in time;
B) Determining the presence or absence of a genetic mutation in an individual cell belonging to the population of cells obtained from the patient, the genetic mutation selected from Table 4 and Table 5;
C) Calculating the ratio of the individual cells with the genetic mutation present to individual cells without the genetic mutation present; and
D) Comparing the ratio obtained in step C between the at least two points in time.

13. The method of claim 12 wherein the proliferative disease or disorder is cancer.

14. The method of claim 13 wherein the cancer is MM.

15. The method of claim 12 wherein the proliferative disease or disorder is WM.

16. The method of claim 12 wherein the population of cells from a patient is an isolated population comprising B cells.


SEQUENCE LISTING (1 sequence)

SEQ ID NO: 1 - DNA, 4201 bp, Homo sapiens
  ref <308>: DATABASE ACCESSION NUMBER: NCBI GenBank/NT_011109
  ref <309>: DATABASE ENTRY DATE: 2006-03-01
  ref <313>: RELEVANT RESIDUES: (24484441 )..(24488641)
aaggaaggagagtgtcttgtccaggttcaaacggctagcacccagcactgagcccagcgtccccaattctcaatcatccattccccttagttctcaaaaaggtggaggctgggggaaaataaataaaattctttattacatcctgatcacacagtagaaatggagattaaataagaacgaggagaaagcaggaccctttccctccactcctcagatcccacaccctgaccaataaataccctccctggagacccagaagtcccagtcccaatatagtccagactgaagaatcttgggctgaccccctcgttttgcagaggagggaaactgaggcccagagaaccacccagcaagttcgtggctcggggcccagcagctctcctctggcctcccctgaagacccttgaggcggcatccgcgtggctggactcacacctggacgcggtagcccccggtccgccgccggcaaagcctccgcacgcccacccagtacagcgtcaacatggccacccagtagcccacgtaggcgccggcccccgcggccaagtggtaggcctcggctgcgcgggaagggccgctccagtcggccctggcctcgtgtgctacgctgcggaccaggcccccaagcagcagcagcgcccagagcgccaggggcagcagagggacgtagttagcggccagcttccgccggcccgaggtgccccagccactctggttcatggtgactagcgccaggaacttggcaggcaggaggccacacatgtagaggggcgcgtagagcgacagaagcaccatgcgcaggcagccccgcagccaggccgcgaaggccgccttggccagtgccacgccctgcacgcacagcagcacccacagcagcgcccaagggcggcccgcgtagaacagacgcagcacagtggccgccacgaagaaggggaacaggccggagaccaccgcctcgtaggtcatccacgcatggtgccggtgccaccagagcgcgttgtacagccactcacggaagtacgacttggaccagcgtgtctgctggctcagccaccgcaggaaggacgagggcgtctctgagtagcagcgggacctggaggtgtacctgcacgggggcgaggaatgagggcatcatcgcgtgctccctggggcctgggcaggattctggaggcagaaatgaccactgtggacggccactgggggcgagtttcttaacctctctaggcctcagtgttctcatgtgtagaatagggtggataatggttcctaggcctggacgcggtggctcactcctgtaatcccagcactttgggaggctgagggataaggagttcaagaccagcctgggcaacatagcaagaccccatctctactaaataaataaataaataaataaataagcaccaatgggtgggatgcaatggctcatgcctgtaatcccagcactttaggaggctgaggtagggggatcacttgaggctaagagtttgagatcagcctaggcaacatagtgagaccccatctctaaaaaaaaaaaaaaaaaaaaaaaattagccgggcgtggtggcctgtagtcccagctatttgggaggctgagatgggggaatcacttgagctcaggagtttgaggctgcagtgagccatgatcgtgcccctgcactccagcctgagcctgagcaacacagcaagactctatctaagaaaaaaaaaaaggctctcataagacaaggatactgtaaggattgaatgaaattaaacgccaagtgcttaggaccagtagatagtatttgctcaataaatgttagctactaccgtttttactatgactattgctgttaccattactattgctgttactaatatcatgttggttactgacactattactaatactatcactaacatattactaatactaccatgcttactattactggcaggccctatagggaggagcaagatgggtgtggttgctacccagggtagtctatgtattagaacttctcaagggaaccatcaggcctccttcctgttttgtggcagacatcattaatcaatccctgcatggtttccagccaagcccacacaagaggagaatcctgggcattacttgacagtgtccagactccttcccctgtgtcacttggcatcctcagtggcctccctgcttccgaccttgccttttgtagtctgtacccaaaacggctgccagagggatcccattacaacgtgtcacttctctgcttaaaaccctcccatggctccctcatcttagtcagagtccttagacctataagcctttcatgagtggaccactccttaactctccatcctgctctcctaatactcccccagttcagtgctccagccacactattctcagccttgagccccatcctacctcaggcactccacttaacacttcacccccaaccccaaaacttcctatccaccacccttgcccggcattatttatctccataacacatatcatgaacatactgtatgttctacttatttatttcttgtgtgtctccccaactgatgtaatcaattggtgtcctgtctgcctattgctgtatccccgcagcttaaacattctaggcacctagtgaatatttgttgagtgaattaatgcatggattcccctggattatcccaagtttcctctgggtatttcagtggacctggctttgcagaaatcactctcccctgaacttcacttggtctagatcatcctcccctgagtctcatggaattacttgtcttctcttgaagaacttactagagttcaggtgctcctcttctctcacttgtttctttctccgtggctctctcactagttagatcccactctatccatttgtctcttgcccttcctacttcactccccactgtgctaccttggtctccatgacagaaccaactcaccctatctcaattctaggtggaattctctaccctcaccttgcctctcttggtagagttcaccgcattccatggttcaatgcaaatgccagccttgtcccttatagtatcttcccctcttccctaatatctcacttggtagagtccattggctcaatgcaaatggcccctctgcccattatggtatcctcctctctcctctaaagtctcacttgatagagcccattgtctggtgagttactttggatttggagaactctcagcatgcacacacgctaggatattcattggcctccacacatacccgaccacctggtcccctcagcttacttggtagcataacccatgctgagcatgcggttggtgaggtgccggtcatccccaaaagtacagtgggtacccaggaacttctggttgtaccaggcctcaagaaactgctgcaagaggttattcctatataggcctgggtggaggggaggtgtgaaagagtgtcagcctctgctgtcaccaacaccaactccaatgctgtttccagccccaaccccatctccagttccaaccccatcctcagtcatcataatctcaatctcagcactatctccaatcccatctccagcccgaatcctgtcctcaacctcatacgcactccaacttgcctgtcattgaactatacccactgtgactgcaaacctgcccaccccagtctcatcaccaatcccatatccattctcagccccatccaaaacccactgcaacctcaaacctggctccaactctgtctttgactccaagcctatcccatcttcttccccagccccagccccaatcgtcatccatatcctttttccaacctcactccccacaccaaactcagcttgagcccaagcagcatcctcaccgccagcttccagttttatcccatccccaattcctgccctgctgcatctttgttcccaaccccagtcacatcctcagccccatccggcttcccttctccctttccaccccatccacagccctcccaatctctgcccctgcttacctagaggaccgctgatgcaggatacacagtggaagtagctctgacaagcccgctccacattgaaggctacccagtatcgcaggctgcttaggaagctgacccaggagtccagagggttaaggatccgcacgtccccaccaacagcccctacccgggggtcctcgtccagtacccgcacgagctccagcagtgccatggggtccaaccttgtgtccgagtcacagacctgta